(19) United States
(12) Patent Application Publication
(10) Pub. No.: US 2023/0130055 A1
(43) Pub. Date: Apr. 27, 2023

(54) PHARMACEUTICAL COMPOSITIONS WITH ENHANCED STABILITY PROFILES

(71) Applicant: AQUESTIVE THERAPEUTICS, INC., Warren, NJ (US)

(72) Inventors: Stephen Paul Wargacki, Pittstown, NJ (US); Rajesh Kumar Kainthan, Tappan, NY (US); Vincent Buono, Basking Ridge, NJ (US); Alexander Mark Schobel, Vero Beach, FL (US); Michael Koons, York, PA (US); Michael Goodrich, Leesburg, VA (US); Gregory Tsodikov, East Windsor, NJ (US)

(73) Assignee: AQUESTIVE THERAPEUTICS, INC., WARREN, NJ (US)

(21) Appl. No.: 17/971,282

(22) Filed: Oct. 21, 2022

Related U.S. Application Data
(60) Provisional application No. 63/271,001, filed on Oct. 22, 2021.

(51) Int. Cl. A61K 9/70; A61K 47/36; A61K 47/02; A61K 47/10; A61K 47/26; A61K 31/137

(52) U.S. Cl. CPC A61K 9/7007; A61K 47/36; A61K 47/02; A61K 47/10; A61K 47/26; A61K 31/137

(57) ABSTRACT

Soluble drug delivery films can exhibit an enhanced stability.

### PRIORITY CLAIM

This application claims priority to U.S. Provisional Patent Application No. 63/271,001, filed Oct. 22, 2021, which is incorporated by reference in its entirety.

### FIELD OF THE INVENTION

The invention relates to pharmaceutical methods and compositions especially suitable for oral delivery. The film product can include a film forming polymer, an active ingredient, and a desiccant. The composition can also include excipients including one or more of a stabilizer, an antioxidant, permeation enhancer, or an adrenergic receptor interacter.

### BACKGROUND

While active pharmaceutical ingredients may be administered in various forms, film delivery can provided superior organoleptic properties products and improve the physical and chemical stability of alternative dosage forms. However, this delivery method can be associated with slow drug release and permeability, film brittleness and poor stability profiles. There is a need for improved formulations that demonstrate improved dissolution rates and improved stability while delivering an effective amount of an active pharmaceutical ingredient.

### SUMMARY OF THE INVENTION

In general, a pharmaceutical composition with enhanced dissolution can include an active ingredient, a film forming polymer including a starch ether, and a desiccant.

In certain embodiments, the starch ether can be a hydroxyalkyl ether of a starch.

In certain embodiments, the hydroxyalkyl ether of a starch can be a hydroxypropyl ether of a starch.

In certain embodiments, the film forming polymer can be a pea starch.

In certain embodiments, the desiccant can include a silica. In certain embodiments, the desiccant can include a fumed silica or a mesoporous silica. In certain embodiments, the film forming polymer and the desiccant can have a ratio of 10:1 to 2:1 by weight.

In certain embodiments, the active ingredient can include 0.1% to 80% of the composition by weight.

In certain embodiments, the pharmaceutical composition can include a stabilizer. In certain embodiments, the stabilizer can include a chelating agent. In certain embodiments, the pharmaceutical composition further includes an antioxidant. In certain embodiments, the stabilizer can include an ion exchange resin. The resin can be a cation exchange resin.

In certain embodiments, the pharmaceutical composition can include a permeation enhancer. In certain embodiments, the pharmaceutical composition including a permeation enhancer can include an adrenergic receptor interacter.

In certain embodiments, the permeation enhancer can include eugenol.

In certain embodiments, the pharmaceutical composition can include a processing solvent. The processing solvent can be an organic processing solvent.

In certain embodiments, the processing solvent can include one or more of ethanol, acetone, acetonitrile, t-butanol, methanol, 1-propanol, isopropanol, tetrahydrofuran, acetaldehyde, dioxane, or methylisocyanide.

In certain embodiments, the processing solvent can include at least 20% ethanol. In certain embodiments, the processing solvent can include at least 30% ethanol. In certain embodiments, the processing solvent can include at least 40% ethanol. In certain embodiments, the processing solvent can include at least 50% ethanol.

In certain embodiments, the pharmaceutical composition can include a plasticizer. In certain embodiments, the plasticizer can include a polyol. In certain embodiments, the plasticizer can include a pentatol. In certain embodiments, the plasticizer can include a sucralose; sugar alcohols such as sorbitol, mannitol, or xylitol.

In certain embodiments, the pharmaceutical composition can include a viscosity builder.

In certain embodiments, the viscosity builder can include gelatin, xantham gum, ethyl cellulose, hydroxy propyl cellulose, methyl cellulose, microcrystalline cellulose, chitosan, natural gums, polyvinyls, crosslinked polymers, or other synthetic polymers. In certain embodiments, the pharmaceutical composition can include a surfactant. In certain embodiments, the surfactant can include Labrasol®. In certain embodiments, the surfactant can include GMO.

In certain embodiments, the pharmaceutical composition can include an esterase inhibitor. In certain embodiments, the esterase inhibitor can include NaF. In certain embodiments, the pharmaceutical composition can include a sweetener. In certain embodiments, the sweetener can include sucralose. In certain embodiments, the sweetener can include Magnasweet.

In certain embodiments, the pharmaceutical composition can include a flavoring agent.

In certain embodiments, the pharmaceutical composition can include a coloring agent.

In general a pharmaceutical composition for delivering a pharmaceutical composition with enhanced stability can include an active ingredient, a pH modifier including HCl, and a plasticizer including a non-reducing sugar.

In certain embodiments, the non-reducing sugar can be a polyol. In certain embodiments, the non-reducing sugar can be a pentatol. In certain embodiments, the non-reducing sugar can be a xylitol.

In certain embodiments, the pH modifier can result in a formulation pH of 2.5 to 3.5 and plasticizer has a ratio of 1:20 to 1:8 by weight.

In general, a pharmaceutical film product can include an active ingredient, a stabilizer, a plasticizer, and the film product can have a small volume disintegration value in the range of about 1 to about 240 seconds as measured according to a small volume disintegration assay.

In certain embodiments, the film product can have a small volume disintegration time in the range of about 2 to about 30 seconds. In certain embodiments, the film product can have a small volume disintegration time in the range of about 2 to about 10 seconds.

In general, a method of making a pharmaceutical composition with enhanced stability can include forming a composition having a dissolution profile in the range of about 1 to about 60 seconds as measured according to a small volume disintegration assay.

In certain embodiments, the film product can have a partial immersion dissolution value in the range of about 2 to about 30 seconds as measured according to a small volume disintegration assay. In certain embodiments, the film product can have a partial immersion dissolution value in the range of about 2 to about 10 seconds as measured according to a small volume disintegration assay.

In general, a method of making a pharmaceutical formulation with an enhanced dissolution rate can include providing an active ingredient incorporating a desiccant including mesoporous silica and applying a film forming polymer including a pea starch.

In general, a method of making a pharmaceutical formulation with enhanced stability can include providing an active ingredient, incorporating a pH modifier that results in formulation pH of 2.5 to 3.5, and incorporating a plasticizer including xylitol.

In general, a method of stabilizing transmucosally delivered epinephrine can include administering a pharmaceutical composition including an active ingredient, a pH modifier results in a formulation pH of 2.5 to 3.5, a desiccant including mesoporous silica, and a plasticizer including xylitol; and achieving an effective plasma concentration of a pharmaceutically active form of epinephrine in less than 1 hour. In certain embodiments, the pH modifier can be HCl.

In certain embodiments, the active ingredient can include a prodrug of epinephrine.

In certain embodiments, the composition can include a degradant. The degradant can be a hydrolysis product of the prodrug of epinephrine.

In certain embodiments, the degradant can be a portion of the delivered active ingredient delivered to a subject.

In certain embodiments, the degradant level can be present at about 3.5% of more at the end of 6 months.

In certain embodiments, the degradant level can be present at about 2.3% or more at the end of 12 months.

In certain embodiments, the degradant level can be present at about 2.4% or more at the end of 24 months.

In certain embodiments, the degradant can have a degradation rate of about 2.2×10⁻³%.

In certain embodiments, the degradant can maintain a shelf life for storage at 25° C. for at least 3 years.

In certain embodiments, the degradant can maintain a shelf life for storage at 25° C. for at least 4 years.

In certain embodiments, the degradant can maintain a shelf life for storage at 25° C. for at least 5 years.

In certain embodiments, the rate of degradant growth can be substantially unchanged for at least 3 months.

In certain embodiments, the rate of degradant growth can be substantially unchanged for at least 5 months.

Other aspects, embodiments, and features will be apparent from the following description, the drawings, and the claims.

### BRIEF DESCRIPTION OF THE DRAWINGS

FIG. 1 depicts disintegration data of DSF and DESF formulations.

FIG. 2 shows average percent DSF and DESF permeated from films.

FIGS. 3A and 3B shows hydrolysis data across formulations.

FIGS. 4A and 4B show drug release of dipivefrin.

FIGS. 5A and 5B show tissue permeation as function of SVD and PID.

FIG. 6 shows effect of pH on formulation stability.

FIG. 7 shows ex vivo tissue permeation data.

FIG. 8 shows epinephrine concentration over time following administration of the formulations.

FIG. 9 shows median epinephrine Tmax from formulations.

FIG. 10 shows mean change in systolic blood pressure.

FIG. 11 shows a hydrolysis product reaction rate as a function of temperature.

### DETAILED DESCRIPTION OF THE INVENTION

In general, soluble drug delivery films can exhibit an enhanced stability. According to the present formulations and methods, stability of 24 months and greater have been achieved. For example, the drug can include prodrugs of an active material, for example an ester of epinephrine, an acidifying agent, a solvent system, a desiccant, an antioxidant, a polymer system, and an ion exchange resin. Exemplary compositions include an epinephrine soluble film, a dipivefrin soluble film (DSF), and a diisobutyryl epinephrine soluble film (DESF). Administering a pharmaceutically active ingredient can include administering a prodrug. Delivery of drugs or pharmaceuticals transdermally or transmucosally can require that the prodrug, drug, active or pharmaceutical alone or in combination permeate or otherwise cross at least one biological membrane partially or completely in an effective and efficient manner. For example, a method of treating a medical condition in a human subject can include administering a composition including a prodrug and a permeation enhancer from a matrix and the permeation enhancer promoting permeation of the prodrug through a mucosal tissue to achieve an effective plasma concentration of a pharmaceutically active form of the prodrug in the human subject in less than one hour. The acidifying agent can be selected to provide an optimal pH, promote formulation stability, while not contributing to mucosal irritation. The solvent system used to manufacture the film can be optimized to reduce overall aqueous loading of the liquid intermediate and the residual moisture in the films. The desiccant and antioxidant can be provided to reduce moisture damage, and oxidative damage. The polymer system can be designed to minimize polymer derived oxidation degradants and reduce disintegration time. The formulations herein allow for reduced thickness of the film and film coating of up to 50%, thereby reducing disintegration time and hastening drug release. A resin can be incorporated to enhance stability, for example, by selectively sequestering free sodium.

The amount of pharmaceutically active component to be used depends on the desired treatment strength and the composition of the layers, although preferably, the pharmaceutical component comprises from about 0.001% to about 99%, more preferably from about 0.003 to about 75%, and most preferably from about 0.005% to about 50% by weight of the composition, including, more than 0.005%, more than 0.05%, more than 0.5%, more than 1%, more than 5%, more than 10%, more than 15%, more than 20%, more than 30%, about 50%, more than 50%, less than 50%, less than 30%, less than 20%, less than 15%, less than 10%, less than 5%, less than 1%, less than 0.5%, less than 0.05%, or less than 0.005%. The amounts of other components may vary depending on the drug or other components but typically these components comprise no more than 90%, no more than 50%, preferably no more than 30%, and most preferably no more than 15% by total weight of the composition.

The thickness of the film may vary, depending on the thickness of each of the layers and the number of layers. As stated above, both the thickness and amount of layers may be adjusted in order to vary the erosion kinetics. Preferably, if the composition has only two layers, the thickness ranges from 0.005 mm to 2 mm, preferably from 0.01 to 1 mm, and more preferably from 0.1 to 0.5 mm, including greater than 0.1 mm, greater than 0.2 mm, about 0.5 mm, greater than 0.5 mm, less than 0.5 mm, less than 0.2 mm, or less than 0.1 mm. The thickness of each layer may vary from 10 to 90% of the overall thickness of the layered composition, and preferably varies from 30 to 60%, including greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, or less than 10%. Thus, the preferred thickness of each layer may vary from 0.01 mm to 0.9 mm, or from 0.03 to 0.5 mm.

As one skilled in the art will appreciate, when systemic delivery, e.g., transmucosal or transdermal delivery is desired, the treatment site may include any area in which the film is capable of delivery and/or maintaining a desired level of pharmaceutical in the blood, lymph, or other bodily fluid. Typically, such treatment sites include the oral, esophageal, aural, ocular, anal, nasal, and vaginal mucosal tissue, as well as, the skin. If the skin is to be employed as the treatment site, then usually larger areas of the skin wherein movement will not disrupt the adhesion of the film, such as the upper arm or thigh, are preferred.

While the pharmaceutical composition can adhere to mucosal tissues, which are wet tissues by nature, it can also be used on other surfaces such as skin or wounds. The pharmaceutical film can adhere to the skin if prior to application the skin is wet with an aqueous-based fluid such as water, saliva, wound drainage or perspiration. The film can adhere to the skin until it erodes due to contact with water by, for example, rinsing, showering, bathing or washing.

### Film-Forming Polymer

The film-forming polymer or polymers may be a water soluble, water swellable, water miscible, water dispersible, or a combination of one or more either water soluble, water swellable, water miscible, or water dispersible polymers. The film-forming polymer may include cellulose or a cellulose derivative or a starch. It may also include a hydroxyalky ether of a starch, or a hydroalkyl ether of a starch, or a hydroxypropyl ether of a starch. The starch can be a pea starch. Specific examples of a film-forming polymer can include a PVP K90. It can also include a Lycoat® RS. Film-forming polymers can be water soluble polymers which include, but are not limited to, polyethylene oxide (PEO), hydroxypropylmethyl cellulose (HPMC), hydroxyethyl cellulose (HEC), hydroxypropyl cellulose (HPC), polyvinyl pyrrolidone (PVP), carboxymethyl cellulose (CMC), polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water miscible polymers or water dispersible polymers include, but are not limited to, ethyl cellulose, hydroxypropyl ethyl cellulose, cellulose acetate phthalate, hydroxypropyl methyl cellulose phthalate, hydroxypropylmethylcellulose acetate succinate (“HPMCAS”), or combinations thereof.

The polymer system can be structured to minimize polymer derived oxidation degradants and reduce disintegration time. Reducing the thickness of a film coating can reduce disintegration time and thus hasten drug release.

The film-forming polymer or combination of film-forming polymer polymers can be about 5% to about 50% by weight of the pharmaceutical composition. For example, a single film-forming polymer can be about 5% to about 50% w/w of the pharmaceutical composition. A second or additional film-forming polymer can be about 5% to about 50% w/w of the pharmaceutical composition by weight. In certain embodiments, a combination of two or more film-forming polymers can be about 5% to about 50% w/w of the pharmaceutical composition. For example, a film-forming polymer or combination of film-forming polymers can be about 5%, about 10%, about 15%, about 20%, about 25%, about 30%, about 35%, about 40%, about 45%, or about 50% w/w of the pharmaceutical composition.

Additives may be included in the films. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). As used herein, the term “stabilizer” means an excipient capable of preventing aggregation or other physical degradation, as well as chemical degradation, of the active pharmaceutical ingredient, another excipient, or the combination thereof.

Stabilization of compositions can help protect components of a composition against a number of degradation pathways, including trans-esterification and hydrolysis, for example. The compositions and methods described herein can address any of these pathways, or address a combination of the pathways by preventing these mechanisms. For example, a prodrug can form degradation products via hydrolysis or trans-esterification to form intermediate prodrugs, a variant or derivative of a first prodrug, or an active pharmaceutical ingredient.

The prodrug can include a compound of formula (I), wherein

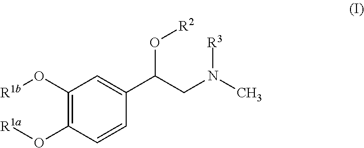

each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof.

In certain embodiments, R² and R³ are H and each R^{1a} and R^{1b}, independently, can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

For example, a diester of epinephrine can undergo hydrolysis to a mono-ester or non-ester form, or combinations thereof. In another example, a triester can undergo hydrolysis to a di-ester, a mono-ester, or non-ester form, or combinations thereof. Transesterification can lead to mixed ester prodrug.

Useful additives can include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), synthetic gums lecithin, pectin, locust bean, starch, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

The additional components can range up to about 80%, desirably about 0.005% to 50% and more desirably within the range of 1% to 20% based on the weight of all composition components, including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, or less than 1%. Other additives can include anti-tacking, flow agents and opacifiers, such as the oxides of magnesium aluminum, silicon, titanium, etc. desirably in a concentration range of about 0.005% to about 15% by weight and desirably about 0.02% to about 2% based on the weight of all film components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, or less than 0.02%. In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols, Erythritol, threitol, xylitol, mannitol, sorbitol, galactitol, fucitol, isomalt, maltitol, lactitol, sorbitol, sodium diethyl sulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition including greater than 0.5%, greater than 1%, greater than 1.5%, greater than 2%, greater than 4%, greater than 5%, greater than 10%, greater than 15%, about 20%, greater than 20%, less than 20%, less than 15%, less than 10%, less than 5%, less than 4%, less than 2%, less than 1%, and less than 0.5%. There may further be added compounds to improve the texture properties of the film material such as animal or vegetable fats, desirably in their hydrogenated form. The composition can also include compounds to improve the textural properties of the product. Other ingredients can include binders which contribute to the ease of formation and general quality of the films. Non-limiting examples of binders include starches, maltodextrin, natural gums, pregelatinized starches, gelatin, polyvinylpyrrolidone, methylcellulose, sodium carboxymethylcellulose, ethylcellulose, polyacrylamides, polyvinyloxoazolidone, or polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

Surface tension reducing agents, anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform films. Agents or components which perform this function can be referred to as densification or densifying agents. As described above, entrapped air or undesired components may lead to non-uniform films. Any other optional components described in commonly assigned U.S. Pat. Nos. 7,425,292 and 8,765,167, referred to above, also may be included in the films described herein.

The pharmaceutical films described herein may be formed via any desired process. Suitable processes are set forth in U.S. Pat. Nos. 8,652,378, 7,425,292 and 7,357,891, which are incorporated by reference herein. In one embodiment, the film dosage composition is formed by first preparing a wet composition, the wet composition including a polymeric carrier matrix and a therapeutically effective amount of a pharmaceutically active component. The wet composition is cast into a film and then sufficiently dried to form a self-supporting film composition. The wet composition may be cast into individual dosages, or it may be cast into a sheet, where the sheet is then cut into individual dosages.

The pharmaceutical composition can adhere to a mucosal surface, such as the mouth, the vagina, organs, or other types of mucosal surfaces. The composition carries a pharmaceutical, and upon application and adherence to the mucosal surface, offers a layer of protection and delivers the pharmaceutical to the treatment site, the surrounding tissues, and other bodily fluids. The composition provides an appropriate residence time for effective drug delivery at the treatment site, given the control of erosion in aqueous solution or bodily fluids such as saliva, and the slow, natural erosion of the film concomitant or subsequent to the delivery.

In certain embodiments, a pharmaceutical composition has a suitable nontoxic, nonionic alkyl glycoside having a hydrophobic alkyl group joined by α linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; Q) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced transmucosal delivery, wherein the formulation of the compound with the transmucosal delivery-enhancing agents provides for increased bioavailability of the compound in blood plasma of a subject. Penetration enhancers have been described in J. Nicolazzo, et al., J. of Controlled Disease, 105 (2005) 1-15, which is incorporated by reference herein.

### Kinetics of Erodibility and Erosion Time

The residence time of the composition depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water soluble organic and inorganic salts; by using crosslinking agents such as glyoxal with polymers such as hydroxyethyl cellulose for partial crosslinking; or by post-treatment irradiation or curing, which may alter the physical state of the film, including its crystallinity or phase transition, once obtained. These strategies might be employed alone or in combination in order to modify the erosion kinetics of the film. Upon application, the pharmaceutical composition film adheres to the mucosal surface and is held in place. Water absorption softens the composition, thereby diminishing the foreign body sensation. As the composition rests on the mucosal surface, delivery of the drug occurs. Residence times may be adjusted over a wide range depending upon the desired timing of the delivery of the chosen pharmaceutical and the desired lifespan of the carrier. Generally, however, the residence time is modulated between about a few seconds to about a few days. Preferably, the residence time for most pharmaceuticals is adjusted from about 5 seconds to about 24 hours. More preferably, the residence time is adjusted from about 5 seconds to about 30 minutes. In addition to providing drug delivery, once the composition adheres to the mucosal surface, it also provides protection to the treatment site, acting as an erodable bandage. Lipophilic agents can be designed to slow down erodability to decrease disintegration and dissolution.

It is also possible to adjust the kinetics of erodibility of the composition by adding excipients which are sensitive to enzymes such as amylase, very soluble in water such as water soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the composition.

In some embodiments, the film dosage may be capable of dispersing and dissolving at a rate of between about 1 to about 30 minutes, for example, about 1 to about 20 minutes, or more than 1 minute, more than 5 minutes, more than 7 minutes, more than 10 minutes, more than 12 minutes, more than 15 minutes, more than 20 minutes, more than 30 minutes, about 30 minutes, or less than 30 minutes, less than 20 minutes, less than 15 minutes, less than 12 minutes, less than 10 minutes, less than 7 minutes, less than 5 minutes, or less than 1 minute. Sublingual dispersion rates may be shorter than buccal dispersion rates.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight, including greater than 5%, greater than 10%, greater than 15%, greater than 20%, greater than 30%, greater than 40%, greater than 50%, greater than 60%, and greater than 70%, about 70%, less than 70%, less than 60%, less than 50%, less than 40%, less than 30%, less than 20%, less than 10% or less than 5% by weight.

### Small Volume Disintegration (SVD)

Dissolution testing is a core performance test in pharmaceutical development and quality control. Dissolution testing has more and more evolved to establish relationships with in vivo performance or with manufacturing Critical Quality Attributes (CQA) in the scope of Quality by Design (QbD). The overall goal is to better control product performance within the life cycle of a product. For this purpose, the use of the classical USP dissolution working conditions using a one liter vessel with basket (respectively USP1) and paddle (respectively USP2) are well established and are used as the first choice for development of a new dissolution method. Nevertheless, limitations coming from the amount of material available, analytical sensitivity, lack of discrimination or biorelevance may warrant the use of non-compendial methods. In particular, in early phase development, during screening of drug candidates, formulation is often developed for studies in animals and dissolution should be ideally conducted using media simulating the gastrointestinal environment as well as in volumes in line with the animal physiology. Another case in which a classical method is not well suited is for low dose drugs or if the analytical method is not sensitive enough to detect the amount of dissolved drug precisely due to low concentration of the drug in the formulation. To overcome those problems the concept of small-volume disintegration arose recently due to the possibility of using smaller sample sizes and smaller volumes of media, offering various advantages in view of substance and material consumption and can serve as a valuable tool for dosage form screening or formulation selection.

Disintegration/residence time for dosages applied via sublingual administration is an important design element for the dosages residing within the sublingual space. Currently, USP <701> is employed to determine disintegration time, with results being obtained for dosage disintegration following exposure to aqueous media under mechanized agitation, with an endpoint being understood as the formation of “non-palpable mass.” To better understand and predict in vivo dosage disintegration, a limited volume, non-agitated methodology (referred to as Small Volume Disintegration or “SVD”) can be applied to produce more differentiating disintegration method highlighting solvation of dosages from a single side and edges. In in this case, SVD utilizes a dosage applied to the surface of an aqueous media volume within a Petri dish.

The SVD time is described as the time (in seconds) taken by a film to disperse when it comes to contact with water or saliva. To a petri dish containing 20 mL water or saliva, a film strip is immersed. The film absorbs water and starts to swell. After some time the film bursts and disperses. The time take by the film to disperse or disintegrate is noted as the small volume disintegration time.

For example, a volume can include 20 mL, Media: Sterile Water or Simulated Saliva. SVD can also highlight visualization and timing until of dosage swelling, bursting (considered end point), and disintegration. SVD times have been used as a variable for DOE-driven product design of novel and generic film products, and predictive models for in vivo film disintegration time.

### Fatty Acids

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference herein. There are two families of essential fatty acids that are metabolized in the human body: ω-3 and ω-6 polyunsaturated fatty acids (PUFAs). If the first double bond is found between the third and the fourth carbon atom from the ω carbon, they are called ω-3 fatty acids. If the first double bond is between the sixth and seventh carbon atom, they are called ω-6 fatty acids. PUFAs are further metabolized in the body by the addition of carbon atoms and by desaturation (extraction of hydrogen). Linoleic acid, which is a ω-6 fatty acid, is metabolized to γ-linolenic acid, dihomo-γ-linolinic acid, arachidonic acid, adrenic acid, tetracosatetraenoic acid, tetracosapentaenoic acid and docosapentaenoic acid. α-Linolenic acid, which is a ω-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na⁺K⁺-APTase pump and the fatty acids with increasing degrees of cis-unsaturation had higher potencies. See, Pomposiello, S. I. et al., Hypertension 31:615-20 (1998), which is incorporated by reference herein. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Feddersen, C. O. et al., J. Appl. Physiol. 68(5):1799-808 (1990); and see, Spannhake, E. W., et al., J. Appl. Physiol. 44:397-495 (1978) and Wicks, T. C. et al., Circ. Res. 38:167-71 (1976), each of which is incorporated by reference herein.

Many studies have reported effects of EPA and DHA on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F, et al., Hypertension 21:22-8 (1993), and Tagawa, H. et al., J Cardiovasc Pharmacol 33:633-40 (1999), each of which is incorporated by reference herein. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference herein. Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50(4):164-70 (2007), which is incorporated by reference herein.

### Adrenergic Receptor Interacters

The adrenergic receptors (or adrenoceptors) are a class of G protein-coupled receptors that are a target of catecholamines, especially norepinephrine (noradrenaline) and epinephrine (adrenaline). Epinephrine (adrenaline) interacts with both α- and β-adrenoceptors, causing vasoconstriction and vasodilation, respectively. Although a receptors are less sensitive to epinephrine, when activated, they override the vasodilation mediated by β-adrenoceptors because there are more peripheral al receptors than β-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The α1-adrenoreceptor is known for smooth muscle contraction, mydriasis, vasoconstriction in the skin, mucosa and abdominal vicera and sphincter contraction of the gastrointestinal (GI) tract and urinary bladder. The α1-adrenergic receptors are member of the G_{q} protein-coupled receptor superfamily. Upon activation, a heterotrimeric G protein, G_{q}, activates phospholipase C (PLC). The mechanism of action involves interaction with calcium channels and changing the calcium content in a cell. For review, see Smith R. S. et al., Journal of Neurophysiology 102(2): 1103-14 (2009), which is incorporated by reference herein. Many cells possess these receptors.

α1-adrenergic receptors can be a main receptor for fatty acids. For example, saw palmetto extract (SPE), widely used for the treatment of benign prostatic hyperplasia (BPH), has been reported to bind α1-adrenergic, muscarinic and 1,4-dihydropyridine (1,4-DHP) calcium channel antagonist receptors. See, Abe M., et al., Biol. Pharm. Bull. 32(4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to α1-adrenergic, muscarinic and 1,4-DHP calcium channel antagonist receptors.

In certain embodiments, a permeation enhancer can be an adrenergic receptor interacter. An adrenergic receptor interacter refers to a compound or substance that modifies and/or otherwise alters the action of an adrenergic receptor. For example, an adrenergic receptor interacter can prevent stimulation of the receptor by increasing, or decreasing their ability to bind. Such interacters can be provided in either short-acting or long-acting forms. Certain short-acting interacters can work quickly, but their effects last only a few hours. Certain long-acting interacters can take longer to work, but their effects can last longer. The interacter can be selected and/or designed based on, e.g., on one or more of the desired delivery and dose, active pharmaceutical ingredient, permeation modifier, permeation enhancer, matrix, and the condition being treated. An adrenergic receptor interacter can be an adrenergic receptor blocker. The adrenergic receptor interacter can be a terpene (e.g. volatile unsaturated hydrocarbons found in the essential oils of plants, derived from units of isoprenes) or a C3-C22 alcohol or acid, preferably a C7-C18 alcohol or acid. In certain embodiments, the adrenergic receptor interacter can include farnesol, linoleic acid, arachidonic acid, docosahexanoic acid, eicosapentanoic acid, and/or docosapentanoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

The C3-C22 alcohol or acid can be an alcohol or acid having a straight C3-C22 hydrocarbon chain, for example a C3-C22 hydrocarbon chain optionally containing at least one double bond, at least one triple bond, or at least one double bond and one triple bond; said hydrocarbon chain being optionally substituted with C₁₋₄ alkyl, C₂₋₄ alkenyl, C₂₋₄ alkynyl, C₁₋₄ alkoxy, hydroxyl, halo, amino, nitro, cyano, C₃₋₅ cycloalkyl, 3-5 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl.

Fatty acids with a higher degree of unsaturation are effective candidates to enhance the permeation of drugs. Unsaturated fatty acids showed higher enhancement than saturated fatty acids, and the enhancement increased with the number of double bonds. See, A. Mittal, et al. Status of Fatty Acids as Skin Penetration Enhancers—A Review, Current Drug Delivery, 2009, 6, pp. 274-279, which is incorporated by reference herein. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than a fatty acid which has double bond at an odd number position. Cis-unsaturation in the chain can tend to increase activity.

An adrenergic receptor interacter can be a terpene. Hypotensive activity of terpenes in essential oils has been reported. See, Menezes I. A. et al., Z. Naturforsch. 65c:652-66 (2010), which is incorporated by reference herein. In certain embodiments, the permeation enhancer can be a sesquiterpene. Sesquiterpenes are a class of terpenes that consist of three isoprene units and have the empirical formula C₁₅H₂₄. Like monoterpenes, sesquiterpenes may be acyclic or contain rings, including many unique combinations. Biochemical modifications such as oxidation or rearrangement produce the related sesquiterpenoids.

An adrenergic receptor interacter can be an unsaturated fatty acid such as linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in perfumes and flavors. It has been reported that farnesol selectively kills acute myeloid leukemia blasts and leukemic cell lines in preference to primary hemopoietic cells. See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both farnesol and N-acetyl-S-trans, trans-farnesyl-L-cysteine (AFC), a synthetic mimic of the carboxyl terminus of farnesylated proteins inhibited vasoconstriction in rat aortic rings.

In certain embodiments, an interacter can be an aporphine alkaloid. For example, an interacter can be a dicentrine.

In general, an interacter can also be a vasodilator or a therapeutic vasodilator. Vasodilators are drugs that open or widen blood vessels. They are typically used to treat hypertension, heart failure and angina, but can be used to treat other conditions as well, including glaucoma for example. Some vasodilators that act primarily on resistance vessels (arterial dilators) are used for hypertension, and heart failure, and angina; however, reflex cardiac stimulation makes some arterial dilators unsuitable for angina. Venous dilators are very effective for angina, and sometimes used for heart failure, but are not used as primary therapy for hypertension. Vasodilator drugs can be mixed (or balanced) vasodilators in that they dilate both arteries and veins and therefore can have wide application in hypertension, heart failure and angina. Some vasodilators, because of their mechanism of action, also have other important actions that can in some cases enhance their therapeutic utility or provide some additional therapeutic benefit. For example, some calcium channel blockers not only dilate blood vessels, but also depress cardiac mechanical and electrical function, which can enhance their antihypertensive actions and confer additional therapeutic benefit such as blocking arrhythmias.

Vasodilator drugs can be classified based on their site of action (arterial versus venous) or by mechanism of action. Some drugs primarily dilate resistance vessels (arterial dilators; e.g., hydralazine), while others primarily affect venous capacitance vessels (venous dilators; e.g., nitroglycerine). Many vasodilator drugs have mixed arterial and venous dilator properties (mixed dilators; e.g., alpha-adrenoceptor antagonists, angiotensin converting enzyme inhibitors), such as phentolamine.

It is more common, however, to classify vasodilator drugs based on their primary mechanism of action. These classes of drugs, as well as other classes that produce vasodilation, include: alpha-adrenoceptor antagonists (alpha-blockers); Angiotensin converting enzyme (ACE) inhibitors; Angiotensin receptor blockers (ARBs); beta₂-adrenoceptor agonists (β₂-agonists); calcium-channel blockers (CCBs); centrally acting sympatholytics; direct acting vasodilators; endothelin receptor antagonists; ganglionic blockers; nitrodilators; phosphodiesterase inhibitors; potassium-channel openers; renin inhibitors.

In general, the active or inactive components or ingredients can be substances or compounds that create an increased blood flow or flushing of the tissue to enable a modification or difference (increase or decrease) in transmucosal uptake of the API(s), and/or have a positive or negative heat of solution which are used as aids to modify (increase or decrease) transmucosal uptake.

### Desiccant

A desiccant is a compound that is designed to safeguard pharmaceutical products from moisture damage by absorbing moisture by physical adsorption or by chemical reaction, thereby improving the stability of the product. A suitable desiccant can include a silica, fumed silica or a mesoporous silica. Examples of desiccants include silicon dioxide variants such as Cab-o-Sil and Syloid 244FP. The desiccant can be 1-15% by weight of the pharmaceutical composition. For example, it can be 2%, 3%, 4%, 5%, 6%, 7%, 8%, 9%, 10%, 11%, 12%, 13%, 14%, or 15% w/w of the pharmaceutical composition.

### Stabilizer

A stabilizer is a material designed to help the active pharmaceutical ingredient (API) maintain the desirable properties of the product or pharmaceutical composition until it is consumed by the patient or otherwise take effect. For example, gelling agents can stabilize liquid dosage forms, such as suspensions and emulsions. Examples include methacrylic acid copolymers, cellulose acetate, alginate and polystyrene sulfonic acid. A stabilizer can also be used in connection with a pH modifier and a plasticizer to prevent or reduce degradation or hydrolysis of the pharmaceutical composition. Stabilizers may also be classified as antioxidants, ion scavengers, sequestrants, pH modifiers, emulsifiers and/or surfactants, and UV stabilizers. A stabilizer can protect a composition or a component of a composition from a degradation pathway, for example, trans-esterification and/or hydrolysis by preventing any of these mechanisms or a combination of these mechanisms. Examples include polystyrene sulfonic acid.

A stabilizer can be an ion exchange resin. It can be a cation exchange resin. It can be structured to scavenge ions. It can scavenge basic ions. It can be an Amberlite® resin, such as AmberLite® HPR1100 Na Ion, Amberlite® IR-120(H) or Amberlite® IRP64. It can be chelating agent. It can be egtazic acid (EGTA), Ethylenediaminetetraacetic acid (EDTA), or citric acid, tartaric acid, ethylene diamine tetra(methylene phosphonic acid), 2-[Bis(carboxymethyl)amino]acetic acid, or (ethylene glycol-bis(O-aminoethyl ether)-N,N,N′,N′-tetraacetic acid). A single stabilizer can be about 0.1% to about 50% w/w of the pharmaceutical composition, preferably about 0.1% to about 100% w/w of the pharmaceutical composition. For example, it can be about 1%, 2%, 3%, 4%, 5%, 6%, 7%, 8%, 9%, or 10% w/w of the pharmaceutical composition.

In certain embodiments, stabilizers can include antioxidants, which can prevent unwanted oxidation of materials, sequestrants, which can form chelate complexes and inactivating traces of metal ions that would otherwise act as catalysts, emulsifiers and surfactants, which can stabilize emulsions, ultraviolet stabilizers, which can protect materials from harmful effects of ultraviolet radiation, UV absorbers, chemicals absorbing ultraviolet radiation and preventing it from penetrating the composition, quenchers, which can dissipate the radiation energy as heat instead of letting it break chemical bonds, or scavengers which can eliminate free radicals formed by ultraviolet radiation.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetyl serotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, o-phenanthroline, and citrate. “Detergents” include, but are not limited to, SDS and sodium lauroyl sarcosyl. “Chaotropes” include, but are not limited to guandinium hydrochloride, isothiocyanate, urea, and formamide. As discussed herein, stabilizers can be present in 0.0001%-50% by weight, including greater than 0.0001%, greater than 0.001%, greater than 0.01%, greater than 0.1%, greater than 1%, greater than 5%, greater than 10%, greater than 20%, greater than 30%, greater than 40%, greater than 50%, less than 50%, less than 40%, less than 30%, less than 20%, less than 10%, less than 1%, less than 0.1%, less than 0.01%, less than 0.001%, or less than 0.0001% by weight.

### Antioxidant

An antioxidant is a molecule capable of decreasing or preventing the oxidation of other molecules or otherwise improve the shelf life of pharmaceuticals. Antioxidants are efficient excipients that delay or inhibit the oxidation of organic and inorganic molecules, preventing the degradation. Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by a linkages), cyclogalactins (5 or more β-D-galactopyranose units linked at the 1,4 positions by R linkages), cycloaltrins (5 or more α-D-altropyranose units linked at the 1,4 positions by a linkages), and combinations thereof. pH modifiers include acids (e.g., tartaric acid, citric acid, lactic acid, fumaric acid, phosphoric acid, ascorbic acid, acetic acid, succininc acid, adipic acid,maleic acid, ethylene diamine tetra(methylene phosphonic acid), 2-[Bis(carboxymethyl)amino]acetic acid, (ethylene glycol-bis(O-aminoethyl ether)-N,N,N′,N′-tetraacetic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

In an exemplary pharmaceutical formulation, an antioxidant can be about 0.1% to about 20% of the pharmaceutical composition by weight. For example, it can be about 0.1%, 0.2%, 0.3%, 0.4%, 0.5%, 1%, 2%, 3%, 4%, 5%, 6%, 7%, 8%, 9%, 10%, 12%, 14%, 16%, 18% or 20% w/w of the pharmaceutical composition. Examples of antioxidants include EGTA, EDTA, citric acid, L-cysteine, and caffeic acid.

### pH Modifier

A pH modifier excipient is used in the pharmaceutical industry due to its antioxidant properties and ability to help maintain the stability of pharmaceutical and can also be used as preservatives. For pH-modification, the addition of a base or an acid is often preferred over the use of buffers. The pH modifier can result in a formulation of pH 2.5 to 6, preferably 2.5 to 4.0, and more preferably from 2.5 to 3.5. The pH of the formulation balances permeability of the active through a mucosal surface and promote formulation stability while not contributing to mucosal irritation. The pH modifier can be a straight acid. Examples of a pH modifier include hydrochloric acid, phosphoric acid, hydrofluoric acid, and citric acid. In certain embodiments, the pH modifier can be an acid reducer.

In certain circumstances, the film compositions can further contain a buffer so as to control the pH of the film composition. Any desired level of buffer may be incorporated into the film composition so as to provide the desired pH level encountered as the pharmaceutically active component is released from the composition. The buffer is preferably provided in an amount sufficient to control the release from the film and/or the absorption into the body of the pharmaceutically active component. In some embodiments, the buffer may include sodium citrate, citric acid, bitartrate salt and combinations thereof.

### Processing Solvent

The processing solvent incorporates an organic/aqueous solvent system to displace and reduce bound water, thereby reducing the overall aqueous loading of the liquid intermediate. The solvent can be hydroorganic or hydroalcoholic, for example, a mixture including at least an alcohol or organic solvent and water. In some embodiments, it can be an ethanol/water mixture. The processing solvent can include an organic solvent that is more volatile than water. In certain circumstances, the organic solvent can form an azeotrope with water. The processing solvent can include 2% to 70% water, 5% to 60% water, or 10% to 50% water. For example, the processing solvent can be a 50:50 ethanol/water w/w mixture. It other embodiments, the processing solvent can include acetone or acetonitrile. In other embodiments, the processing solvent can include one or more of t-butanol, methanol, 1-propanol, isopropanol, tetrahydrofuran, acetaldehyde, dioxane, dichloromethane, or methylisocyanide. The processing solvent can also be a mixture of one or more of the foregoing solvents.

### API Loading

The active pharmaceutical ingredient (API) can be provided with sufficiently high API loads to limit the dosage form size while optimizing the dose for each API. In one example, the API load can be greater than 8%, greater than 10%, greater than 12%, greater than 14%, greater than 16%, greater than 18%, greater than 20%, greater than 22%, or greater than 24% w/w. The API load can be between 2% and 40% w/w, or between 5% and 30% w/w.

### Permeation Enhancer

A permeation enhancer can be provided to improve the permeation of a drug or API through a surface, e.g. a mucosal surface. An example is eugenol. The permeation enhancer can be provided in a concentration of greater than 1%, greater than 2%, greater than 5%, greater than 10%, greater than 12%, greater than 14%, greater than 16%, greater than 18%, greater than 20%, greater than 25%, greater than 30%, greater than 35%, or greater than 40% w/w.

A particular class of permeation enhancer can improve the uptake and bioavailability of the pharmaceutically active component in vivo. In particular, when delivered to the mouth via a film, the permeation enhancer can improve the permeability of the pharmaceutically active component through the mucosa and into the blood stream of the subject. The permeation enhancer can improve absorption rate and amount of the pharmaceutically active component by more than 5%, more than 10%, more than 20%, more than 30%, more than 40%, more than 50%, more than 60%, more than 70%, more than 80%, more than 90%, more than 100%, more than 150%, about 200% or more, or less than 200%, less than 150%, less than 100%, less than 90%, less than 80%, less than 70%, less than 60%, less than 50%, less than 40%, less than 30%, less than 20%, less than 10%, or less than 5%, or a combination of these ranges, depending on the other components in the composition.

Chemical penetration enhancers are substances that control the permeation rate of a coadministered drug through a biological membrane. While extensive research has focused on obtaining an improved understanding of how penetration enhancers might alter intestinal and transdermal permeability, far less is known about the mechanisms involved in buccal and sublingual penetration enhancement.

The buccal mucosa delineates the inside lining of the cheek as well as the area between the gums and upper and lower lips and it has an average surface area of 100 cm². The surface of the buccal mucosa consists of a stratified squamous epithelium which is separated from the underlying connective tissue (lamina propria and submucosa) by an undulating basement membrane (a continuous layer of extracellular material approximately 1-2 μm in thickness). This stratified squamous epithelium consists of differentiating layers of cells which change in size, shape, and content as they travel from the basal region to the superficial region, where the cells are shed. There are approximately 40-50 cell layers, resulting in a buccal mucosa which is 500-600 μm thick.

Structurally the sublingual mucosa is comparable to the buccal mucosa but the thickness of this epithelium is 100-200 μm. This membrane is also non-keratinised and being relatively thinner has been demonstrated to be more permeable than buccal mucosa. Blood flow to the sublingual mucosal is slower compared with the buccal mucosa and is of the order of 1.0 ml/min-1/cm-2.

The permeability of the buccal mucosa is greater than that of the skin, but less than that of the intestine. The differences in permeability are the result of structural differences between each of the tissues. The absence of organized lipid lamellae in the intercellular spaces of the buccal mucosa results in greater permeability of exogenous compounds, compared to keratinized epithelia of the skin; while the increased thickness and lack of tight junctions results in the buccal mucosa being less permeable than intestinal tissue.

The primary barrier properties of the buccal mucosa have been attributed to the upper one-third to one-quarter of the buccal epithelium. Researchers have learned that beyond the surface epithelium, the permeability barrier of nonkeratinized oral mucosa could also be attributed to contents extruded from the membrane-coating granules into the epithelial intercellular spaces.

The intercellular lipids of the nonkeratinized regions of the oral cavity are of a more polar nature than the lipids of the epidermis, palate, and gingiva, and this difference in the chemical nature of the lipids may contribute to the differences in permeability observed between these tissues. Consequently, it appears that it is not only the greater degree of intercellular lipid packing in the stratum corneum of keratinized epithelia that creates a more effective barrier, but also the chemical nature of the lipids present within that barrier.

The existence of hydrophilic and lipophilic regions in the oral mucosa has led researchers to postulate the existence of two routes of drug transport through the buccal mucosa paracellular (between the cells) and transcellular (across the cells).

Since drug delivery through the buccal mucosa is limited by the barrier nature of the epithelium and the area available for absorption, various enhancement strategies are required in order to deliver therapeutically relevant amounts of drug to the systemic circulation. Various methods, including the use of chemical penetration enhancers, prodrugs, and physical methods may be employed to overcome the barrier properties of the buccal mucosa.

A chemical penetration enhancer, or absorption promoter, is a substance added to a pharmaceutical formulation in order to increase the membrane permeation or absorption rate of the coadministered drug, without damaging the membrane and/or causing toxicity. There have been many studies investigating the effect of chemical penetration enhancers on the delivery of compounds across the skin, nasal mucosa, and intestine. In recent years, more attention has been given to the effect of these agents on the permeability of the buccal mucosa. Since permeability across the buccal mucosa is considered to be a passive diffusion process the steady state flux (Jss) should increase with increasing donor chamber concentration (CD) according to Fick's first law of diffusion.

Surfactants and bile salts have been shown to enhance the permeability of various compounds across the buccal mucosa, both in vitro and in vivo. The data obtained from these studies strongly suggest that the enhancement in permeability is due to an effect of the surfactants on the mucosal intercellular lipids.

Fatty acids have been shown to enhance the permeation of a number of drugs through the skin, and this has been shown by differential scanning calorimetry and Fourier transform infrared spectroscopy to be related to an increase in the fluidity of intercellular lipids. Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azone® on the permeability of compounds through oral mucosa. Further, chitosan, a biocompatible and biodegradable polymer, has been shown to enhance drug delivery through various tissues, including the intestine and nasal mucosa.

Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery-Current status and future prospects, Int'l. Journal of Pharmaceutics, 47(2014) 498-506, which is incorporated by reference herein. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium, as well as the emergence of new analytical techniques to study the oral cavity, and the progressing development of in silico models predictive of buccal and sublingual permeation, prospects are encouraging.

In order to deliver broader classes of drugs across the buccal mucosa, reversible methods of reducing the barrier potential of this tissue should be employed. This requisite has fostered the study of penetration enhancers that will safely alter the permeability restrictions of the buccal mucosa. It has been shown that buccal penetration can be improved by using various classes of transmucosal and transdermal penetration enhancers such as bile salts, surfactants, fatty acids and their derivatives, chelators, cyclodextrins and chitosan. Among these chemicals used for the drug permeation enhancement, bile salts are the most common.

In vitro studies on enhancing effect of bile salts on the buccal permeation of compounds is discussed in Sevda Senel, Drug permeation enhancement via buccal route: possibilities and limitations, Journal of Controlled Release 72 (2001) 133-144, which is incorporated by reference herein. That article also discusses recent studies on the effects of buccal epithelial permeability of dihydroxy bile salts, sodium glycodeoxycholate (SGDC) and sodium taurodeoxycholate (TDC) and tri-hydroxy bile salts, sodium glycocholate(GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, or safrole, or a combination thereof. The phytoextract can be an essential oil extract of a clove plant, for example, from the leaf, stem or flower bud of a clove plant. The clove plant can be Syzygium aromaticum. The phytoextract can include 20-95% eugenol, including 40-95% eugenol, including 60-95% eugenol, and for example, 80-95% eugenol. The extract can also include 5% to 15% eugenol acetate. The extract can also include caryophyllene. The extract can also include up to 2.1% α-humulen. Other volatile compounds included in lower concentrations in clove essential oil can be β-pinene, limonene, farnesol, benzaldehyde, 2-heptanone and ethyl hexanoate. Other permeation enhancers may be added to the composition to improve absorption of the drug. Suitable permeation enhancers include natural or synthetic bile salts such as sodium fusidate; glycocholate or deoxycholate and their salts; fatty acids and derivatives such as sodium laurate, oleic acid, oleyl alcohol, monoolein, and palmitoylcarnitine; chelators such as citric acid, tartaric acid, Ethylene diamine tetra(methylene phosphonic acid), 2-[Bis(carboxymethyl)amino]acetic acid, (ethylene glycol-bis(O-aminoethyl ether)-N,N,N′,N′-tetraacetic acid), disodium EDTA, sodium citrate and sodium laurylsulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol®. The permeation enhancer can include phytoextract derivatives and/or monolignols. The permeation enhancer can also be a fungal extract.

### Plasticizer

Plasticizers can be added to the polymers used as film forming agents in order to make the polymer pliable and soft, enhancing the flexibility and plasticity of the films. A plasticizer is often used for solid dosage forms, particularly oral solid dosage forms. Examples include glycerin, propylene glycol, polyethylene glycols (PEG), organic esters such as diethyl esters, dibutyl esters, dibutyl sebacete, citrate esters, triacetin. Other examples include oils or glycerides such as castor oil, acetylated monoglycerides, or fractionated coconut oil. It can be a carbohydrate, polyalcohol or sugar alcohol such as mannitol, sorbitol, xylitol, lactitol, isomalt, maltitol and hydrogenated starch hydrolysates (HSH). The plasticizer can be provided in greater than 2%, greater than 4%, greater than 6%, greater than 8% or greater than 10% w/w.

### Viscosity Modifier

Viscosity modifiers are designed to change the thickness or texture of pharmaceutical ingredients. Viscosity modifiers can include such products as thickeners, texturizers, gelation agents and stiffening agents. Many viscosity modifiers can be used to convert liquids to gels, pastes or powders to aid formulators in creating the ideal product for end users. A viscosity modifier can decrease the thickness of a liquid to improve pour ability and ultimately make it more palatable. Certain examples are natural or synthetic gums, which can be derived from sugars. The viscosity modifier can be provided in greater than 0.2%, greater than 0.4%, greater than 0.6%, greater than 0.8%, greater than 1%, greater than 1.2%, greater than 1.4%, or greater than 1.6%. An example is gelatin, xantham gum, ethyl cellulose, hydroxy propyl cellulose, methyl cellulose, microcrystalline cellulose, chitosan, natural gums, and other synthetic polymers.

### Surfactant

A surfactant is an agent that absorbs to surfaces, interfaces to reduce surface or interfacial tension or is used to lower the surface tension between liquids. Surfactants aid wetting and dispersion of hydrophobic active pharmaceutical ingredients and usually act by reducing the interfacial tension between solids and liquids in suspensions. Surfactants can be nonionic, anionic, cationic, or amphoteric. These surfactants differ in composition and polarity. Examples of suitable surfactants include PEG derivatives, such as ethoxylates, Labrasol®, or Transcutol®, and PEG-fatty acid esters, PEG amine ethers. Examples can also include structure-forming lipids and aliphatic alcohols, i.e., monoglycerides such as glyceryl monooleate (GMO) and phytantriol (PHT).

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C₁₀-C₂₄)-alkyl-dimethylammonium chloride or bromide), (C₁₀-C₂₄)-alkyl-dimethylethylammonium chloride or bromide, (C₁₀-C₂₄)-alkyl-trimethylammonium chloride or bromide (e.g., cetyltrimethylammonium chloride or bromide), (C₁₀-C₂₄)-alkyl-dimethylbenzylammonium chloride or bromide (e.g., (C₁₂-C₁₈)-alkyl-dimethylbenzylammonium chloride), N(C₁₀-C₁₈)-alkyl-pyridinium chloride or bromide (e.g., N—(C₁₂-C₁₆)-alkyl-pyridinium chloride or bromide), N—(C₁₀-C₁₈)-alkyl-isoquinolinium chloride, bromide or monoalkyl sulfate, N—(C₁₂-C₁₈)-alkyl-polyoylaminoformylmethylpyridinium chloride, N—(C₁₂-C₁₈)-alkyl-N-methylmorpholinium chloride, bromide or monoalkyl sulfate, N(C₁₂-C₁₈)-alkyl-N-ethylmorpholinium chloride, bromide or monoalkyl sulfate, (C₁₆-C₁₈)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, allyl ethers, alkyl aryl ethers, sorbitan monostearate, sorbitan monooleate and/or sorbitan monopalmitate.

### Esterase Inhibitor

An esterase inhibitor prevents chemical or enzyme hydrolysis of drugs such as esters, amides and carbamates during analytical manipulation which is desirable to achieve a drug's desired pharmacokinetics. Such hydrolysis occurs through the action of non-specific esterases which are present in blood, plasma and tissues. Examples of such esterase inhibitors include sodium fluoride (NaF), diisopropyl-fluorophosphate (DFP) and 1,5, bis(4-allyl dimethyl ammonium phenyl)-pentan-3-one dibromide (ADAPP), H₂NSO₃⁻, I⁻, SCN⁻, NO₃⁻, NO₂⁻, N₃⁻, I⁻, Br⁻, Cl⁻, SO₄²⁻, S⁻², PO₄³⁻, HPO₄²⁻, H₂PO⁴⁻, HSO⁴⁻, SO₃²⁻, CO³⁻, or C₂O₄².

### Sweeteners

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Sweeteners may be broadly grouped into two categories—nutritive and non-nutritive. Nutritive sweeteners deliver calories and as their name suggests, non-nutritive do not. Non-nutritive sweeteners can be further characterized as bulk (sugar alcohols) and high intensity (artificial). Some examples include aspartame, saccharin, sucralose, Acesulfame K, Magnasweet, Stevia, and sugar alcohols.

### Flavors and Color

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

A flavoring agent can be added to the formulation in an attempt to enhance the organoleptic properties of the formulation or mask its bitterness or other unpleasant taste. Reducing bitterness can be accomplished by balancing with the complementary tastes—sweet, sour and salty via the mechanism of taste/taste interaction—the foundational principle of taste and flavor masking. Once the bitterness has been reduced, pharmaceutical flavoring agents such as orange, grape or mint can be selected based on compatibility with the drug active and excipients, patient demographics, as well as dosing frequency and other quality of life factors. A proper drug formulation requires a robust and balanced base to achieve palatability.

### Coloring Agents

Colorants or coloring agents are mainly used to impart a distinctive appearance to the pharmaceutical dosage forms. Suitable colorants enhance the aesthetic appearance of dosage forms particularly for oral pharmaceutical preparations. The Food Drug and Cosmetic Act of 1938 created three categories of Dyes: (1) FD&C colors—colorants that are certifiable for use in foods, drugs, and cosmetics; (2) D&C colors—dyes and pigments considered safe for use in drugs and cosmetics when in contact with mucous membranes or when ingested; and (3) External D&C colors—colorants, due to their oral toxicity, are not certifiable for use in products intended for ingestion but are considered safe for use in products applied externally. Some examples of widely used colorants in pharmaceuticals include FD&C Blue No. 1—Brilliant Blue, (blue shade), FD&C Blue No. 2—Indigotine, (indigo shade), FD&C Red No. 3—Erythrosine, (pink shade), FD&C Red No. 40—Allura Red, (red shade), FD&C Yellow No. 5—Tartrazine, (yellow shade) and FD&C Yellow No. 6—Sunset Yellow, (orange shade).

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

### Sequence of Permeation Enhancer(s) and Active Pharmaceutical Ingredient(s)

The arrangement, order, or sequence of penetration enhancer(s) and active pharmaceutical ingredient(s)(API(s)) delivered to the desired mucosal surface can vary in order to deliver a desired pharmacokinetic profile. For example, one can apply the permeation enhancer(s) first by a film, by swab, spray, gel, rinse or by a first layer of a film then apply the API(s) by single film, by swab, or by a second layer of a film. The sequence can be reversed or modified, for example, by applying the API(s) first by film, by swab, or by a first layer of a film, and then applying the permeation enhancer(s) by a film, by swab, spray, gel, rinse or by a second layer of a film. In another embodiment, one may apply a permeation enhancer(s) by a film, and a drug by a different film. For example, the permeation enhancer(s) film positioned under a film containing the API(s), or the film containing the API(s) positioned under a film containing the permeation enhancer(s), depending on the desired pharmacokinetic profile.

For example, the penetration enhancer(s) can be used as a pretreatment alone or in combination with at least one API to precondition the mucosa for further absorption of the API(s). The treatment can be followed by another treatment with neat penetration enhancer(s) to follow the at least one API mucosal application. The pretreatment can be applied as a separate treatment (film, gel, solution, swab etc.) or as a layer within a multilayered film construction of one or more layers. Similarly, the pretreatment may be contained within a distinct domain of a single film, designed to dissolve and release to the mucosa prior to release of the secondary domains with or without penetration enhancer(s) or API(s). The active ingredient may then be delivered from a second treatment, alone or in combination with additional penetration enhancer(s). There may also be a tertiary treatment or domain that delivers additional penetration enhancer(s) and/or at least one API(s) or prodrug(s), either at a different ratio relative to each other or relative to the overall loading of the other treatments. This allows a custom pharmacokinetic profile to be obtained. In this way, the product may have single or multiple domains, with penetration enhancer(s) and API(s) that can vary in mucosal application order, composition, concentration, or overall loading that leads to the desired absorption amounts and/or rates that achieve the intended pharmacokinetic profile and/or pharmacodynamic effect.

The film format can be oriented such that no distinct sides, or such that the film has at least one side of a multiple layer film where the edges are co-terminus (having or meeting at a shared border or limit).

The pharmaceutical composition can be a chewable or gelatin based dosage form, spray, gum, gel, cream, tablet, liquid, powder inhalation, or film. The composition can include textures, for example, at the surface, such as microneedles or micro-protrusions. Recently, the use of micron-scale needles in increasing skin permeability has been shown to significantly increase transdermal delivery, including and especially for macromolecules. Most drug delivery studies have emphasized solid microneedles, which have been shown to increase skin permeability to a broad range of molecules and nanoparticles in vitro. In vivo studies have demonstrated delivery of oligonucleotides, reduction of blood glucose level by insulin, and induction of immune responses from protein and DNA vaccines. For such studies, needle arrays have been used to pierce holes into skin to increase transport by diffusion or iontophoresis or as drug carriers that release drug into the skin from a microneedle surface coating. Hollow microneedles have also been developed and shown to microinject insulin to diabetic rats. To address practical applications of microneedles, the ratio of microneedle fracture force to skin insertion force (i.e. margin of safety) was found to be optimal for needles with small tip radius and large wall thickness. Microneedles inserted into the skin of human subjects were reported as painless. Together, these results suggest that microneedles represent a promising technology to deliver therapeutic compounds into the skin for a range of possible applications. Using the tools of the microelectronics industry, microneedles have been fabricated with a range of sizes, shapes and materials. Microneedles can be, for example, polymeric, microscopic needles that deliver encapsulated drugs in a minimally invasive manner, but other suitable materials can be used.

Applicants have found that microneedles could be used to enhance the delivery of drugs through the oral mucosa, particularly with the claimed compositions. The microneedles create micron sized pores in the oral mucosa which can enhance the delivery of drugs across the mucosa. Solid, hollow or dissolving microneedles can be fabricated out of suitable materials including, but not limited to, metal, polymer, glass and ceramics. The microfabrication process can include photolithography, silicon etching, laser cutting, metal electroplating, metal electro polishing and molding. Microneedles could be solid which is used to pretreat the tissue and are removed before applying the film. The drug loaded polymer film described in this application can be used as the matrix material of the microneedles itself. These films can have microneedles or micro protrusions fabricated on their surface which will dissolve after forming microchannels in the mucosa through which drugs can permeate.

The term “film” can include films and sheets, in any shape, including rectangular, square, or other desired shape. A film can be any desired thickness and size. In preferred embodiments, a film can have a thickness and size such that it can be administered to a user, for example, placed into the oral cavity of the user. A film can have a relatively thin thickness of from about 0.0025 mm to about 0.250 mm, or a film can have a somewhat thicker thickness of from about 0.250 mm to about 1.0 mm. For some films, the thickness may be even larger, i.e., greater than about 1.0 mm or thinner, i.e., less than about 0.0025 mm. For example, the film can have dimensions of 10 mm by 10 mm up to 30 mm by 30 mm. A film can be a single layer or a film can be multi-layered, including laminated or multiple cast films. A permeation enhancer and pharmaceutically active component can be combined in a single layer, each contained in separate layers, or can each be otherwise contained in discrete regions of the same dosage form. In certain embodiments, the pharmaceutically active component contained in the polymeric matrix can be dispersed in the matrix. In certain embodiments, the permeation enhancer being contained in the polymeric matrix can be dispersed in the matrix.

Oral dissolving films can fall into three main classes: fast dissolving, moderate dissolving and slow dissolving. Oral dissolving films can also include a combination of any of the above categories. Fast dissolving films can dissolve in about 1 second to about 30 seconds in the mouth, including more than 1 second, more than 5 seconds, more than 10 seconds, more than 20 seconds, and less than 30 seconds. Moderate dissolving films can dissolve in about 1 to about 30 minutes in the mouth including more than 1 minute, more than 5 minutes, more than 10 minutes, more than 20 minutes or less than 30 minutes, and slow dissolving films can dissolve in more than 30 minutes in the mouth. As a general trend, fast dissolving films can include (or consist of) low molecular weight hydrophilic polymers (e.g., polymers having a molecular weight between about 1,000 to 9,000 daltons, or polymers having a molecular weight up to 200,000 daltons). In contrast, slow dissolving films generally include high molecular weight polymers (e.g., having a molecular weight in millions). Moderate dissolving films can tend to fall in between the fast and slow dissolving films.

It can be preferable to use films that are moderate dissolving films. Moderate dissolving films can dissolve rather quickly, but also have a good level of mucoadhesion. Moderate dissolving films can also be flexible, quickly wettable, and are typically non-irritating to the user. Such moderate dissolving films can provide a quick enough dissolution rate, most desirably between about 1 minute and about 20 minutes, while providing an acceptable mucoadhesion level such that the film is not easily removable once it is placed in the oral cavity of the user. This can ensure delivery of a pharmaceutically active component to a user.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug. For example, the pharmaceutically active component can be an ester of epinephrine, for example, dipivefrin. See, e.g., J. Anderson, et al., Site of ocular hydrolysis of a prodrug, dipivefrin, and a comparison of its ocular metabolism with that of the parent compounds, epinephrine, Invest., Ophthalmol. Vis. Sci. July 1980. In certain embodiments, administering the prodrug stimulates one or more adrenergic receptors. In certain embodiments, administering the prodrug may not activate the alpha 1 adrenergic receptor relative to epinephrine. In certain embodiments, the pharmaceutically active form of the prodrug has a Tmax of less than 60 minutes. In certain embodiments, the prodrug has a Tmax of less than 30 minutes. In certain embodiments, the prodrug has a Tmax of less than 15 minutes.

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

For example, the pharmaceutically active component can be buprenorphine, naloxone, acetaminophen, riluzole, clobazam, Rizatriptan, propofol, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, hydrocortisone, predonisolone, dexarnethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, fluocinonide, edaravone, lurasidone, esomeprazole, lumateperone, naldmedine, doxylamine, pyridoxine, diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, thimerosal, phenol, thymol, benzalkonium chloride, benzethonium chloride, chlorhexidine, povidone iodide, cetylpyridinium chloride, eugenol, trimethylammonium bromide, naphazoline nitrate, tetrahydrozoline hydrochloride, oxymetazoline hydrochloride, phenylephrine hydrochloride, tramazoline hydrochloride, thrombin, phytonadione, protamine sulfate, aminocaproic acid, tranexamic acid, carbazochrome, carbaxochrome sodium sulfanate, rutin, hesperidin, sulfamine, sulfathiazole, sulfadiazine, homosulfamine, sulfisoxazole, sulfisomidine, sulfamethizole, nitrofurazone, penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, salicylic acid, podophyllum resin, podolifox, cantharidin, chloroacetic acids, silver nitrate, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir, heparin, insulin, LHRH, TRH, interferons, oligonuclides, calcitonin, octreotide, omeprazone, fluoxetine, ethinylestradiol, amiodipine, paroxetine, enalapril, lisinopril, leuprolide, prevastatin, lovastatin, norethindrone, risperidone, olanzapine, albuterol, hydrochlorothiazide, pseudoephridrine, warfarin, terazosin, cisapride, ipratropium, busprione, methylphenidate, levothyroxine, zolpidem, levonorgestrel, glyburide, benazepril, medroxyprogesterone, clonazepam, ondansetron, losartan, quinapril, nitroglycerin, midazolam versed, cetirizine, doxazosin, glipizide, vaccine hepatitis B, salmeterol, sumatriptan, triamcinolone acetonide, goserelin, beclomethasone, granisteron, desogestrel, alprazolam, estradiol, nicotine, interferon beta 1A, cromolyn, fosinopril, digoxin, fluticasone, bisoprolol, calcitril, captorpril, butorphanol, clonidine, premarin, testosterone, sumatriptan, clotrimazole, bisacodyl, dextromethorphan, nitroglycerine, nafarelin, dinoprostone, nicotine, bisacodyl, goserelin, and granisetron. In certain embodiments, the pharmaceutically active component can be epinephrine, a prodrug of epinephrine, a benzodiazepine such as diazepam or lorazepam or alprazolam.

### Epinephrine/Dipivefrin Examples

In one example, a composition including epinephrine or its salts or esters (such as dipivefrin) can have a biodelivery profile similar to that of epinephrine administered by injection, for example, using an EpiPen®. Epinephrine or its prodrug can be present in an amount of from about 0.01 mg to about 100 mg per dosage, for example, at a 0.1 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. In another example, a composition including diazepam can have a biodelivery profile similar to that of a diazepam tablet or gel, or better.

Dipivefrin can be present in an amount of from about 0.5 mg to about 100 mg per dosage, for example, at a 0.5 mg, 1 mg, 5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof.

In another example, a composition (e.g., including epinephrine) can have a suitable nontoxic, nonionic alkyl glycoside having a hydrophobic alkyl group joined by α linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; or (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject. The formulation can include approximately the same active pharmaceutical ingredient (API): enhancer ratio as in the other examples for epinephrine.

Administering epinephrine as a prodrug such as dipivefrin confers certain advantages. For one, dipivefrin is lipophilic and therefore has a higher permeation through a mucosa. It also has a longer plasma half-life due to higher protein binding. It is capable of sustained blood levels, and does not interact with α-receptors, therefore minimizing or eliminating unwanted or harmful vasoconstriction.

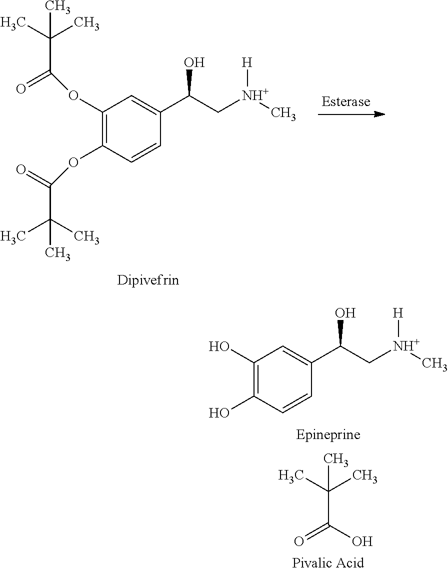

Dipivefrin can be provided as sublingual film in a similar manner as with epinephrine.

A film and/or its components can be water-soluble, water swellable or water-insoluble. The term “water-soluble” can refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water. The composition can include a polymeric matrix. Any desired polymeric matrix may be used, provided that it is orally dissolvable or erodible. The dosage should have enough bioadhesion to not be easily removed and it should form a gel like structure when administered. They can be moderate-dissolving in the oral cavity and particularly suitable for delivery of pharmaceutically active components, although both fast release, delayed release, controlled release and sustained release compositions are also among the various embodiments contemplated.

### Branched Polymers

The pharmaceutical composition film can include dendritic polymers which can include highly branched macromolecules with various structural architectures. The dendritic polymers can include dendrimers, dendronised polymers (dendrigrafted polymers), linear dendritic hybrids, multi-arm star polymers, or hyperbranched polymers.

Hyperbranched polymers are highly branched polymers with imperfections in their structure. However they can be synthesized in a single step reaction which can be an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications. See, e.g., Dendrimers as Drug Carriers: Applications in Different Routes of Drug Administration. J Pharm Sci, VOL. 97, 2008, 123-143, which is incorporated by reference herein.

The dendritic polymers can have internal cavities which can encapsulate drugs. The steric hindrance caused by the highly dense polymer chains might prevent the crystallization of the drugs. Thus, branched polymers can provide additional advantages in formulating crystallizable drugs in a polymer matrix.

Examples of suitable dendritic polymers include poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers or hyperbranched polymers.

Other examples of hyperbranched polymers include poly(amines)s, polycarbonates, poly(ether ketone)s, polyurethanes, polycarbosilanes, polysiloxanes, poly(ester amine)s, poly(sulfone amine)s, poly(urea urethane)s and polyether polyols such as polyglycerols.

A film can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through a controlled drying processes, which include application of heat and/or radiation energy to the wet film matrix to form a visco-elastic structure, thereby controlling the uniformity of content of the film. The controlled drying processes can include air alone, heat alone or heat and air together contacting the top of the film or bottom of the film or the substrate supporting the cast or deposited or extruded film or contacting more than one surface at the same time or at different times during the drying process. Some of such processes are described in more detail in U.S. Pat. Nos. 8,765,167 and 8,652,378, which are incorporated by reference herein. Alternatively, the films may be extruded as described in U.S. Patent Publication No. 2005/0037055 A1, which is incorporated by reference herein.

A polymer included in the films may be water-soluble, water-swellable, water-insoluble, or a combination of one or more either water-soluble, water-swellable or water-insoluble polymers. The polymer may include cellulose, cellulose derivatives or gums. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, films formed from such water-soluble polymers may be sufficiently water-soluble to be dissolvable upon contact with bodily fluids.

Other polymers useful for incorporation into the films include biodegradable polymers, copolymers, block polymers or combinations thereof. It is understood that the term “biodegradable” is intended to include materials that chemically degrade, as opposed to materials that physically break apart (i.e., bioerodable materials). The polymers incorporated in the films can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, polyhydroxy-alkanoates or mixtures thereof. The polymer matrix can include one, two, three, four or more components.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of pharmaceutically active component contained in the composition. Some pharmaceutically active components may only require a few minutes for delivery through the mucosal tissue, whereas other pharmaceutically active components may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods of time, such as up to several hours, which may be desirable for delivery of certain pharmaceutically active components.

### Film Thickness and Size

Desirably, an individual film dosage of the pharmaceutical film can have a suitable thickness, and small size, which is between about 0.0625-3 inch by about 0.0625-3 inch. The film size can also be greater than 0.0625 inch, greater than 0.5 inch, greater than 1 inch, greater than 2 inches, about 3 inches, and greater than 3 inches, less than 3 inches, less than 2 inches, less than 1 inch, less than 0.5 inch, less than 0.0625 inch in at least one aspect, or greater than 0.0625 inch, greater than 0.5 inch, greater than 1 inch, greater than 2 inches, or greater than 3 inches, about 3 inches, less than 3 inches, less than 2 inches, less than 1 inch, less than 0.5 inch, less than 0.0625 inch in another aspect. The aspect ratio, including thickness, length, and width can be optimized by a person of ordinary skill in the art based on the chemical and physical properties of the polymeric matrix, the active pharmaceutical ingredient, dosage, enhancer, and other additives involved as well as the dimensions of the desired dispensing unit. The film dosage should have good adhesion when placed in the buccal cavity or in the sublingual region of the user. Further, the film dosage should disperse and dissolve at a moderate rate, most desirably dispersing within about 1 minute and dissolving within about 3 minutes.

### EXAMPLES

A series of film formulations using diisobutyryl L-epinephrine (DIE) hydrochloride were manufactured changing the excipients and process conditions. The formulation details are provided in the Table 1A. The formulations used in clinical studies are shown in Table 1B. The stability of the formulations was evaluated using 3 day 60° C. thermal stress test. Each excipient change resulted in either significant reductions in total degradants or in the reduction of a specific impurity. The stability data is provided in Table 2.

TABLE 1A
Formulation composition of various film formulations

| | Formulation number | | | | | |
|---|---|---|---|---|---|---|
| | | 45-1-1 or | | | | |
| | 33-1-1 | 45-1-3 | 55-1-1 | 60-1-1 | 93-1-1 | 98-1-1 |
| Formulation components | (Weight %) | (weight %) | (weight %) | (weight %) | (weight %) | (weight %) |
| Diisobutyryl L-epinephrine | 25.000 | 25.000 | 25.000 | 25.000 | 20.000 | 20.00 |
| hydrochloride | | | | | | |
| Pregelatinized hydroxypropyl | N/A | N/A | N/A | N/A | 18.390 | 19.39 |
| pea starch | | | | | | |
| Hydroxy Ethyl Starch | N/A | N/A | N/A | N/A | 2.000 | N/A |
| Polyvinylpyrrolidone | 20.000 | 23.000 | 23.000 | 23.000 | 25.00 | 25.00 |
| Hydroxypropyl Methyl | 10.500 | 11.245 | 11.245 | 10.245 | N/A | N/A |
| Starch | | | | | | |
| Polyethylene oxide | 8.205 | 8.505 | 7.505 | 8.505 | N/A | N/A |
| Glycerol Monooleate | 1.000 | 1.000 | 1.000 | 1.000 | 1.00 | 1.00 |
| Silicon dioxide | N/A | N/A | 2.000 | N/A | 5.00 | 5.00 |
| PEG 400 | 1.000 | 1.000 | N/A | 1.000 | N/A | N/A |
| Xanthan Gum | 1.000 | 1.000 | 1.000 | 1.000 | 1.00 | 1.00 |
| Eugenol | 17.040 | 17.040 | 17.040 | 17.040 | 13.70 | 13.70 |
| citric acid | 4.060 | N/A | N/A | N/A | N/A | N/A |
| Hydrochloric acid (1N) | N/A | 1.200 | 1.200 | 1.200 | 1.20 | 1.20 |
| sucralose | 1.750 | 1.750 | 1.750 | 1.750 | 1.75 | 1.75 |
| Labrasol ® | 1.000 | 1.000 | 1.000 | 1.000 | 1.00 | 1.00 |
| Sodium Fluoride | 1.185 | 0.000 | N/A | N/A | 0.700 | 0.70 |
| EDTA | N/A | N/A | N/A | 1.000 | 1.00 | 1.00 |
| Polacrylex Resin | N/A | N/A | N/A | N/A | N/A | 1.00 |
| Mint Flavor NAT WONF SD | 8.000 | 8.000 | 8.000 | 8.000 | 8.00 | 8.00 |
| Magnasweet ™ | 0.250 | 0.250 | 0.250 | 0.250 | 0.25 | 0.25 |
| FD&C Blue #1 | 0.010 | 0.010 | 0.010 | 0.010 | 0.010 | 0.010 |
| Process solvent | water | 45-1-3 | water/ | water/ | Water/ | Water/ |
| | | (water) | ethanol | ethanol | ethanol | ethanol |
| | | 45-1-1 | | | | |
| | | (water/ | | | | |
| | | ethanol) | | | | |

TABLE 1B
Formulations Used In Clinical Study

| | Formulation 1 | Formulation 2 | Formulation 3 | Formulation 4 |
|---|---|---|---|---|
| | % Solid | % Solid | % Solid | % Solid |
| | Composition | Composition | Composition | Composition |
| Component | (% w/w) | (% w/w) | (% w/w) | (% w/w) |
| L-diisobutyryl epinephrine | 12.50 | 25.00 | 25.00 | 30.00 |
| HCl | | | | |
| Lycoat ® RS 780 | 9.08 | 7.58 | 8.63 | 4.24 |
| Polyvinylpyrrolidone | 30.00 | 25.00 | 25.00 | 25.00 |
| Syloid 244FP | 5.00 | 5.00 | 5.00 | 5.00 |
| Glycerol Monooleate, Type | 1.00 | 1.00 | 1.00 | 1.00 |
| 40 | | | | |
| Xanthan Gum | 1.00 | 1.00 | 1.00 | 1.00 |
| Eugenol | 17.04 | 17.04 | 17.04 | 20.45 |
| Hydrochloric acid 10 wt % | 0.667 | 0.667 | 0.320 | 2.485 |
| Sucralose | 1.75 | 1.75 | 1.75 | 1.75 |
| Amberlite ® IRP64 | 1.00 | 1.00 | 1.00 | 1.00 |
| Sodium Fluoride | 0.70 | 0.70 | N/A | 4.00 |
| Xylitol | 10.00 | 4.00 | 1.068 | 0.50 |
| Edetate Disodium, | 1.00 | 1.00 | 0.267 | 1.00 |
| Dihydrate | | | | |
| Labrasol ® | 1.00 | 1.00 | 0.267 | 1.00 |
| Mint Flavor NAT WONF | 8.00 | 8.00 | 2.136 | 1.315 |
| SD | | | | |
| Magnasweet ™ MM 100 | 0.250 | 0.250 | 0.067 | 0.250 |
| FD&C Blue #1 | 0.010 | 0.010 | 0.003 | 0.010 |

TABLE 2
Progressive Stability Improvements With Excipients And Process Solvent

| | | | | | | | | | | | | | | | | % |
|---|---|---|---|---|---|---|---|---|---|---|---|---|---|---|---|---|
| | | | | | Unknown Degradants (% LC) | | | | | | | | | Total | Total | Assay |
| | Stability | DIE | Epi | MIE | RRT | RRT | RRT | RRT | RRT | RRT | RRT | RRT | RRT | Unknown | degradants | loss |
| Lot | condition | (% LC) | % | (%) | 1.66 | 1.71 | 1.74 | 1.78 | 1.81 | 2.23 | 2.39 | 2.53 | 2.79 | degradants | (%) | (%) |
| 33-1-1 | Initial | 105.4 | 0.2 | 1.6 | ND | 0.02 | 0.05 | 0.04 | ND | 0.08 | 0.04 | 0.06 | ND | 0.29 | 2.1 | N/A |
| | 3 days, | 90.1 | 1.2 | 9.8 | ND | 1.28 | ND | ND | 0.20 | 0.40 | 0.03 | 3.58 | ND | 5.49 | 16.5 | −15.3 |
| | 60° C. | | | | | | | | | | | | | | | |
| 45-1-3 | Initial | 103.3 | 0.2 | 1.1 | ND | ND | 0.13 | ND | 0.07 | 0.10 | ND | ND | ND | 0.30 | 1.6 | N/A |
| | 3 days, | 100.0 | 0.5 | 2.9 | ND | ND | 0.94 | ND | 0.22 | 0.34 | 0.11 | 0.32 | 0.31 | 2.24 | 5.6 | −3.3 |
| | 60° C. | | | | | | | | | | | | | | | |
| 45-1-1 | Initial | 108.3 | 0.4 | 1.7 | ND | 0.14 | ND | 0.05 | 0.19 | 0.15 | 0.02 | ND | ND | 0.55 | 2.7 | N/A |
| | 3 days, | 106.4 | 0.7 | 3.4 | ND | 0.61 | 0.03 | 0.09 | 0.42 | 0.34 | 0.08 | 0.33 | ND | 1.92 | 6.0 | −1.9 |
| | 60° C. | | | | | | | | | | | | | | | |
| 55-1-1 | Initial | 100.2 | 0.4 | 1.6 | ND | ND | 0.13 | ND | 0.13 | 0.08 | 0.03 | ND | ND | 0.36 | 2.3 | N/A |
| | 3 days, | 98.2 | 0.6 | 2.6 | ND | 0.25 | 0.04 | ND | 0.33 | 0.19 | 0.11 | 0.13 | 0.16 | 1.23 | 4.4 | −2.0 |
| | 60° C. | | | | | | | | | | | | | | | |
| 60-1-1 | Initial | 104.6 | 0.2 | 1.0 | ND | ND | 0.11 | ND | 0.04 | ND | 0.17 | ND | ND | 0.32 | 1.6 | N/A |
| | 3 days, | 103.0 | 0.4 | 2.6 | ND | 0.30 | ND | ND | 0.22 | 0.12 | 0.10 | 0.16 | 0.20 | 1.11 | 4.1 | −1.6 |
| | 60° C. | | | | | | | | | | | | | | | |
| 93-1-1 | Initial | 114.6 | 0.3 | 1.4 | 0.03 | ND | 0.18 | ND | ND | ND | ND | ND | ND | 0.21 | 1.9 | N/A |
| | 3 days, | 113.5 | 0.4 | 2.8 | 0.04 | ND | 0.19 | 0.04 | 0.04 | 0.03 | ND | ND | ND | 0.34 | 3.5 | −1.1 |
| | 60° C. | | | | | | | | | | | | | | | |
| 98-1-1 | Initial | 109.8 | 0.3 | 1.4 | ND | ND | 0.13 | ND | ND | ND | ND | ND | ND | 0.13 | 1.9 | N/A |
| | 3 days, | 109.5 | 0.3 | 1.7 | 0.03 | ND | 0.18 | 0.03 | 0.05 | ND | ND | ND | ND | 0.28 | 2.3 | −0.4 |
| | 60° C. | | | | | | | | | | | | | | | |

TABLE 3
Effect of NaCl On Film Disintegration Time

| | | Small volume disintegration |
|---|---|---|
| Formulation number | Attributes | (sec) |
| 163-1-1 | DESF | 260 |
| 161-1-1 | DESF with 0.7% NaCl | 222 |
| 159-1-1 | DESF with 2% NaCl | 222 |
| 160-1-1 | DESF with 4% NaCl | 225 |

### Effect of Hydrochloric Acid as Acidifying Agent on Stability

Citric acid is a common acidifying agent used in film formulations (formulation 33-1-1). A mineral acid like hydrochloric acid was used as an acidifying agent (formulation 45-1-3) and the results showed that HCl is a better acidifying agent as it provides better product stability. The use of HCl resulted in reduction of hydrolytic degradants, as well as other unknown degradants both of which led to reduction in assay loss after the 3 day thermal stress test. Other acids like phosphoric acid and hydrofluoric acid were found to be less effective than HCl.

### Effect of pH on Stability

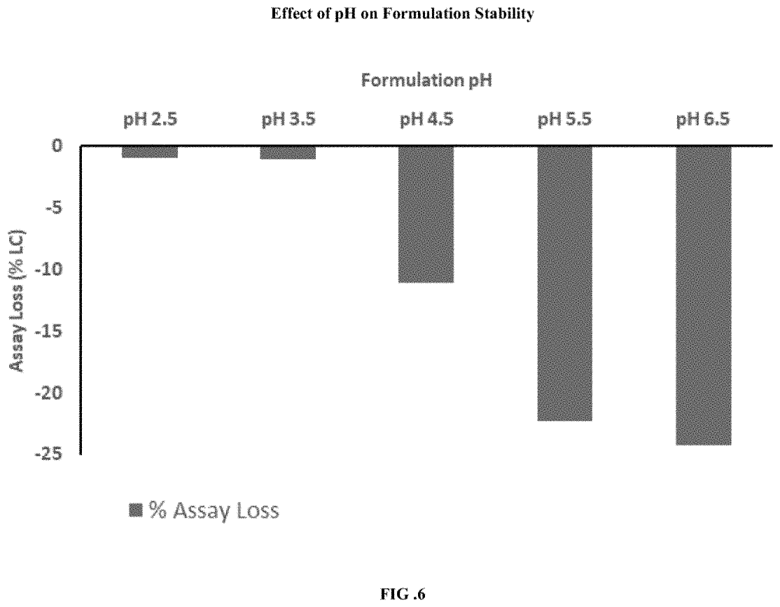

Referring to FIG. 6, a series of formulations were manufactured varying the pH with HCl and the stability was assessed using the 3 day thermal stress test at 60° C. Based on the data a pH range of 2.5 to 3.5 is found to be optimal for stability.

### Effect of Aqueous Organic Solvent as the Process Solvent for Making the Coating Mixture

The formulations were manufactured using an aqueous ethanolic solution for making the coating mixture. The manufactured films were compared for stability at TO and after the thermal stress test. The results showed that the overall unknown degradants and assay loss were further reduced at both the conditions when an organic solvent was used. A 50:50 mixture of ethanol and water were found to be optimal as solvents with reduced ethanol amount led to non-uniform films.

### Effect of Desiccant on Film Stability

Ester prodrugs are generally prone to hydrolysis and loss of assay after storage was attributed to the presence of residual moisture in the films. In order to minimize the presence of free water a desiccant such as silicon dioxide was used. Compared to Cab-O-Sil®, Syloid 244FP used in the formulation 55-1-1, was found to reduce the hydrolysis degradation compared to formulation 45-1-1 which did not have the desiccant. The syloid in the formulation also resulted in films with reduced tackiness.

### Effect of Antioxidant on Film Stability

Some of the unknown degradants in the film were suspected to result from oxidation reaction of the drug. In order to address this several common antioxidants like caffeic acid, L-cysteine, EDTA were tested and EDTA was found to be the most effective one after the thermal stress test. The use of a chelator like EDTA resulted in reduction of hydrolytic degradants, as well as other unknown degradants both of which led to reduction in assay loss after the 3 day thermal stress test.

### Effect of Film Forming Polymers on Stability

In order to reduce the stability issues typically associated with film forming polymers like HPMC and PEO, PVP along with pregelatinized hydroxypropyl pea starch (e.g., Lycoat® RS 780) was employed. This change in film forming polymers resulted in reduced film disintegration time and reduction in unknown impurities leading to reduced assay loss.

### Effect of Resin on Stability

The use of an anionic exchange resin like Amberlite® IRP64 in the film (e.g., formulation 98-1-1) resulted in either reduction or elimination of hydrolysis and unknown degradants which led to reduced assay loss after thermal stress test. The anionic exchange resin might be acting as a scavenging agent of reactive species like alkali or alkaline earth metals which react with the drug.

### Effect of Salt on Film Disintegration

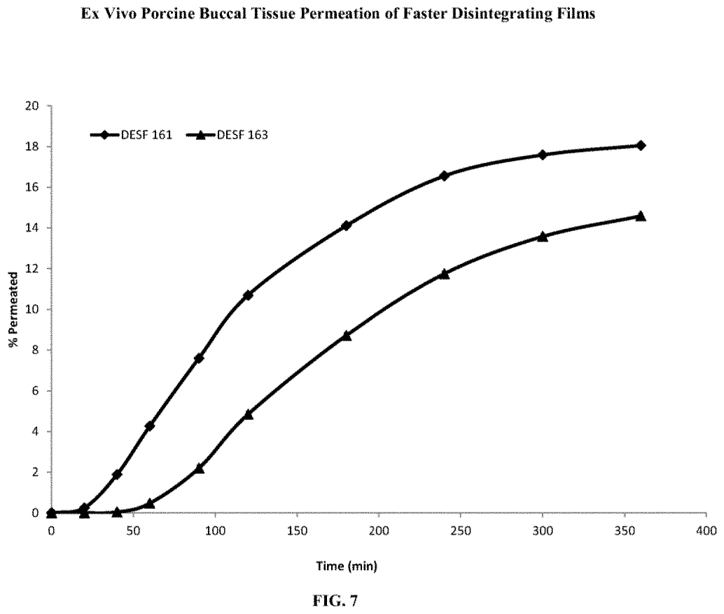

The incorporation of sodium chloride (NaCl) into the film formulation, was found to increase the film disintegration time as tested by small volume disintegration. The data is summarized in Table 3. The faster disintegration films were found to improve the drug permeation as evidenced from the ex vivo buccal permeation data. The data is summarized in FIG. 7. The use of NaCl results in faster release and permeation of the drug from the film resulting in early onset.

### Example 1—Dipivefrin Sublingual Film (DSF) Formulation

TABLE 1
DSF Formulation Example
DSF platform

PVP - 42.56%
PEO - 14.72%
Citric acid - 2.19%
Adrenergic Receptor
Interacter - 10.28%
PEG- 2%
Polysaccharaide - 1%
Surfactant- 1%
Fatty Acid - 1%
Esterase inhibitor - 0.69%
Sugar - 1.75%
Sweetener - 0.25%
Mint flavor 8- %
FD&C Blue #1 0.01%

### Example 2—Diisobutyryl Epinephrine Sublingual Film (DSF) Formulation

TABLE 2
DESF Formulation Example
DESF platform

PVP- 30%
Lycoat ® RS 11.08%
Desiccant - 5%
Resin stabilizer - 1%
Antioxidant - 1%
HCl - 0.67%
Adrenergic receptor interacter -
17.04%
Plasticizer - 8%
Viscosity Builder - 1%
Surfactant - 1%
Fatty acid - 1%
Esterase inhibitor - 0.70%
Sugar - 1.75%
Sweetener - 0.25%
Mint flavor 8- %
FD&C Blue #1 0.01%

### Example 3—pH Modifier Selection

In this Example, the DESF platform utilized a citrate buffer system to produce a target pH range of 3-4. To improve the stability profile of the formulation, the utilization of HCl as an acidifying agent was considered. The impact of HCl as an acidifying agent was compared between the following systems:
1. 33-1-1: PVP/HMPC/PEO system, acidified with a citrate buffer system, aqueous solvent
2. 45-1-3: PVP/HMPC/PEO system, acidified with HCl, aqueous solvent

With HCl as an acidifying agent, the following improvements were noted compared to 33-1-1:
Reduction in the generation of hydrolysis degradants (e.g., epinephrine, monoisobutyryl epinephrine (MIE))
Reduction in overall unknown degradants
Reduction in assay loss

The impact of assay loss with regard to pH was investigated, evaluating a pH range from 2.5-6.5. It was concluded that an operable pH range of pH 2.5-3.5 utilizing could be selected to minimize the impact upon formulation stability.

### Example 4—SVD

Small Volume Disintegration (SVD) was applied to study highlighting solvation of dosages from a single side and edges. In in this case, SVD utilized a dosage applied to the surface of an aqueous media volume within a Petri dish.
SVD was used for 112-1-5 Dipivefrin Sublingual Film.
Average unit dosage weight: 197 mg.
Major compositional information: 14.55% Dipivefrin, 10.28% Eugenol
Film swelling was observed at approximately 2 minutes
Swelling did not expand considerably beyond the original area of the unit dosage.
Film bursting initiated at an average of 12 minutes, 4 seconds (standard deviation: 48.1 seconds).
Film total disintegration was completed at approximately 17 minutes.

### Example 5—Film Disintegration Profiles as a Function of Formulation

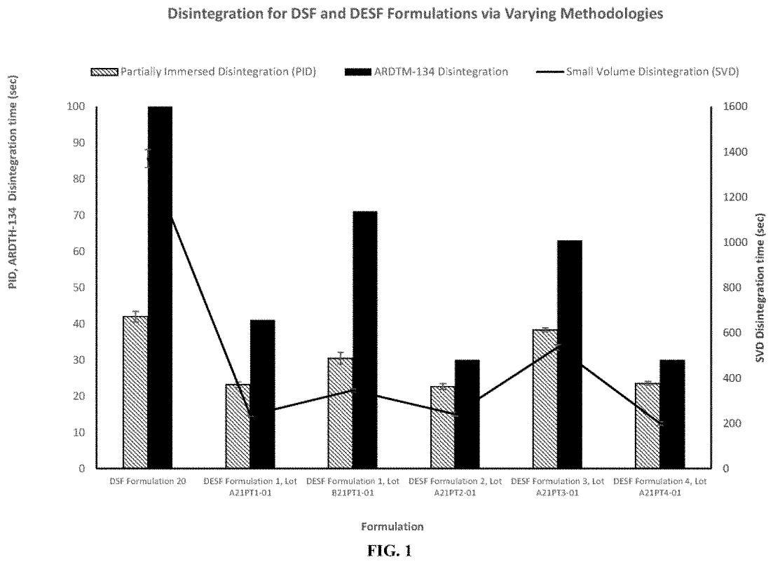

Referring to FIG. 1, the film disintegration profiles are shown as a function of formulation.

TABLE 3
DSF and DESF Disintegration Profiles

| | Partially Immersed | | ARDTM-134 | Small Volume | |
|---|---|---|---|---|---|
| | Disintegration (PID) | | Disintegration | Disintegration | |
| | Disintegration | | Disintegration | Disintegration | (SVD) |
| Formulation | time (sec) | STD EV | time (sec) | time (sec) | STDEV |
| DSF Formulation 20 | 42 | 1.45 | 109 | 1370 | 39.60 |
| DESF Formulation 1, Lot A21PT1-01 | 23 | 0.75 | 41 | 236 | 3.69 |
| DESF Formulation 1, Lot B21PT1-01 | 31 | 1.64 | 71 | 346 | 5.09 |
| DESF Formulation 2, Lot A21PT2-01 | 23 | 0.82 | 30 | 240 | 9.24 |
| DESF Formulation 3, Lot A21PT3-01 | 38 | 0.5 | 63 | 536 | 11.2 |
| DESF Formulation 4, Lot A21PT4-01 | 24 | 0.55 | 30 | 199 | 8.18 |

In this system, DSF formulation used PVP/HMPC/PEO polymer system, 12.5% dipivefrin HCl. DESF formulation used PVP/Lycoat® RS 780 polymer system, 12.5-30% diisobutyryl epinephrine HCl. Disintegration was measured utilizing Partially Immersed Disintegration (PID), USP Disintegration (ARDTM-134), and Small Volume Disintegration (SVD) methodologies. SVD showed significant difference in disintegration times between DSF and DESF platforms.

### Example 6—Drug Release Studies

DSF and DESF systems were designed similarly with the following formulations to study drug release:
DSF: PVP/HMPC/PEO polymer system, 12.5% dipivefrin HCl
DESF: PVP/Lycoat® RS 780 polymer system, 12.5% diisobutyryl epinephrine HCl

Utilizing a polymer/disintegrant excipient approach at a thinner coat weight, DESF platforms showed unexpectedly improved better drug releases profiles. Of the total dosage, the following amounts of either dipivefrin or diisobutyryl epinephrine were released from the film matrix:
DSF: 0.5% at 5 minutes, 21% at 2 hours
DESF: 6% at 5 minutes, 42% at 2 hours

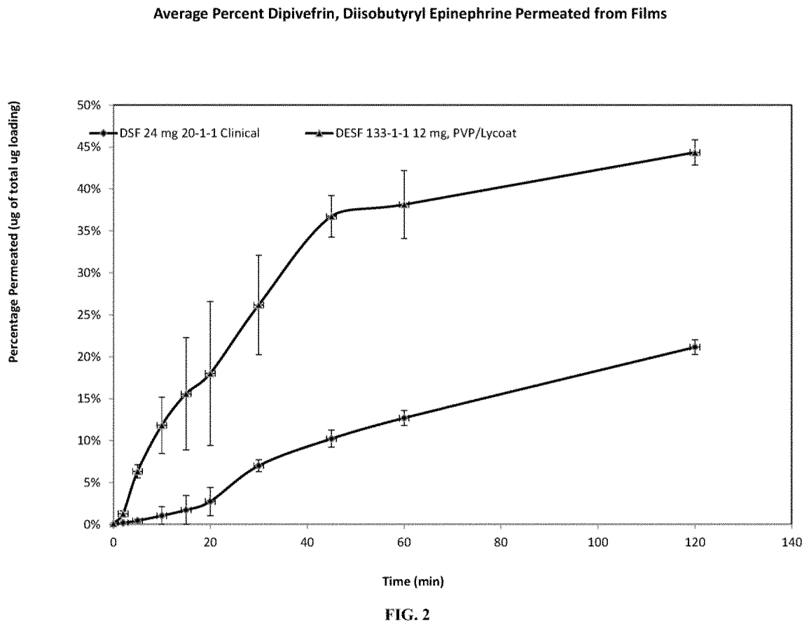

Referring to FIG. 2, both films exhibited biphasic drug release profiles with a rapid drug release from 0 to approximately 40 minutes, and the percent permeation continuing to increase, but gradually tapering after about 40 minutes until 120 minutes.

### Example 7—Effect of Disintegration Time on Tissue Permeation

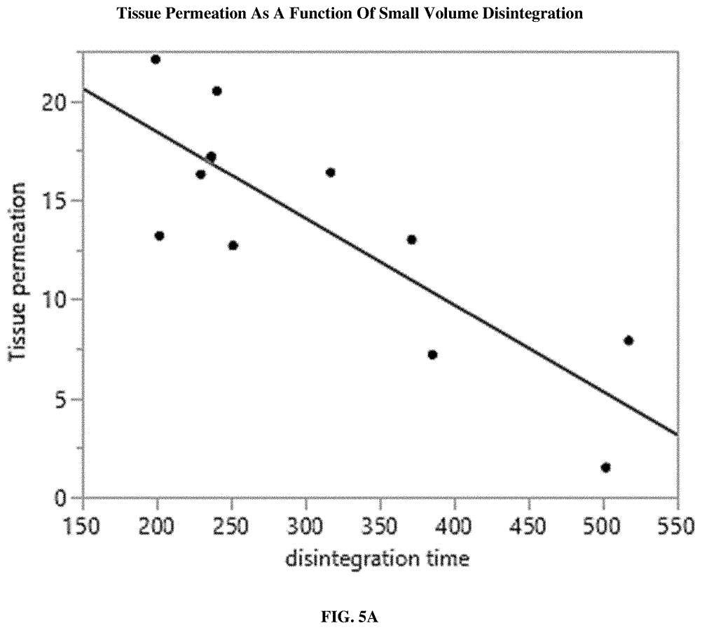

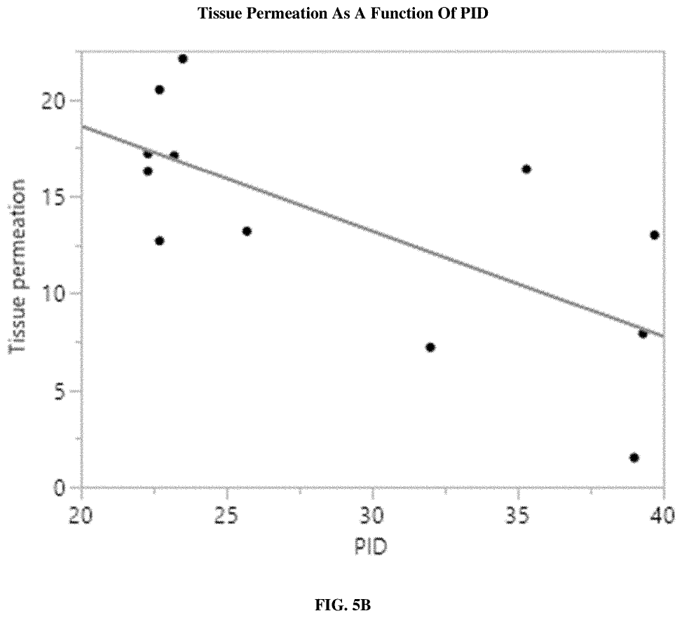

Referring to FIGS. 5A and 5B, the effect of disintegration time on tissue permeation is shown. FIG. 5A was performed using SVD methodologies. FIG. 5B was performed using PID methodologies. With both methods, an increased percentage of API permeated correlated with films exhibiting reduced disintegration times.

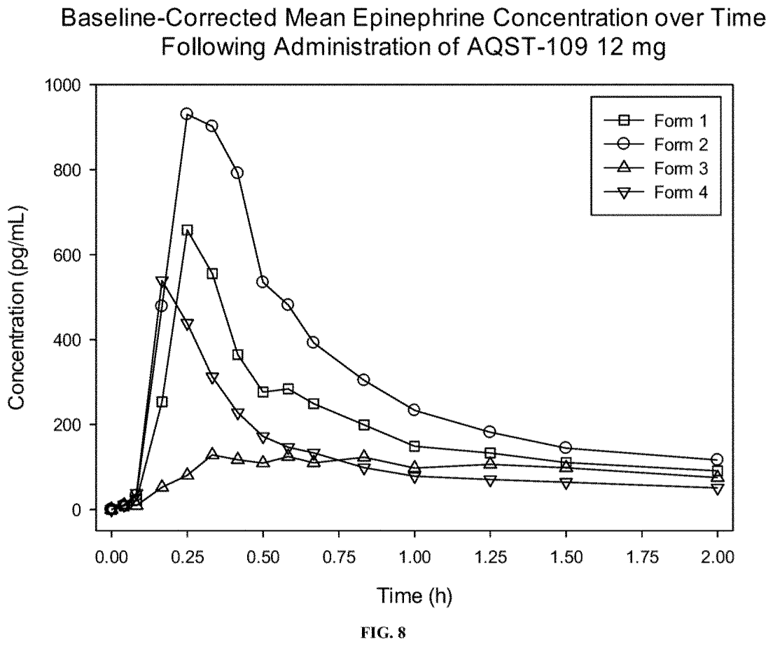

Referring to FIG. 8, a baseline-corrected mean epinephrine concentration was measured over time following administration of the prodrug (12 mg) in Formulations 1, 2, 3 and 4.

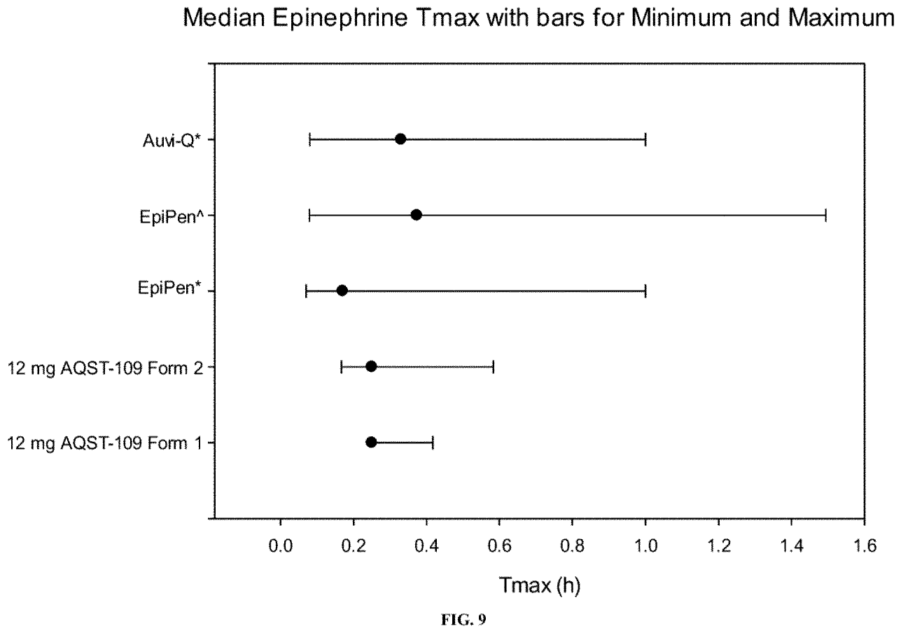

Referring to FIG. 9, median epinephrine Tmax was measured. The results are shown, with bars indicating minimum and maximum.
Tmax (or time to maximum concentration) is a critical parameter for rescue medications
The highest observed Tmax values for Formulations 1, 2, 3 and 4 at 12 mg were below the highest Tmax values for autoinjectors
The median Tmax values for Formulations 1, 2, 3 and 4 were comparable to the known values from the autoinjectors

### Example 8—Polymer to Drug Load Ratio

The following formulations were used to study effective polymer to drug load ratios. Eugenol was used as a permeation enhancer and NaF was used as an esterase inhibitor. Lab scale results were weight normalized. Hydrolysis products and impurities were separately measured.

TABLE 4
Polymer to Drug Load Ratios

| | | Drug | | Sodium |
|---|---|---|---|---|
| Clinical | Lab Scale | Load | Eugenol | Fluoride |
| Formulation | Formulation | (%) | (%) | (%) |
| Form 1 | 126 | 12.5 | 17 | 1 |
| Form 2 | 127 | 25 | 17 | 1 |
| Form 3 | 136 | 25 | 17 | 0 |
| Form 4 | 125 | 30 | 20 | 4 |

Hydrolysis Products (Hydrolysis Method)
   Monoisobutryryl Epinephrine
   Epinephrine: Observed under accelerated conditions (40/75)
Degradants (Impurity Method)
   RRT 1.81: Likely eugenol related.
   RRT 1.91: Likely eugenol related.
   Other Unspecified: Remain near or below quantitation limit after 6 months 25° C.

### Example 9-8 Stability Studies

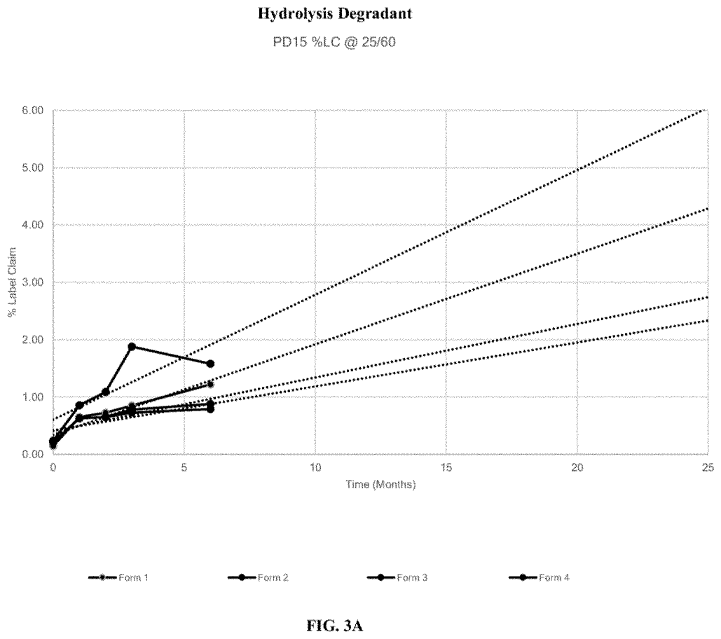

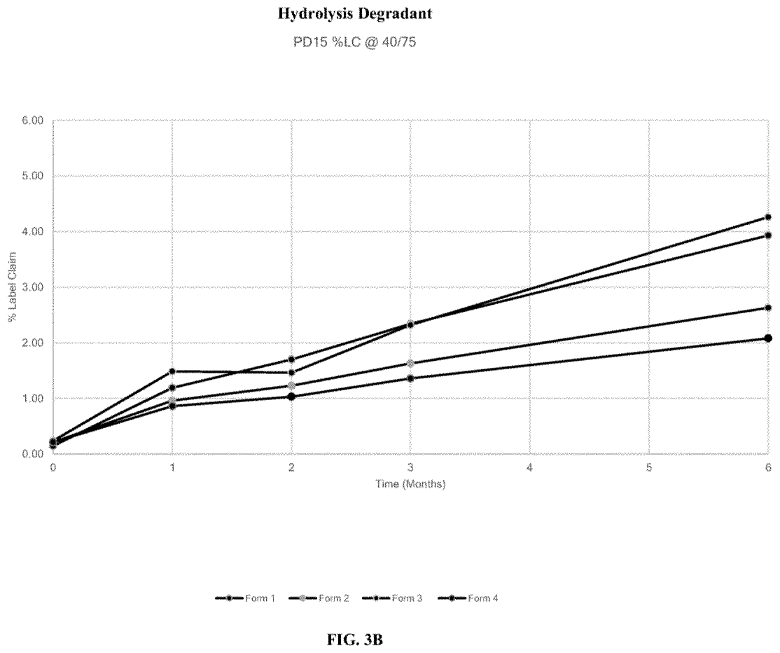

Referring to FIGS. 3A and 3B, these studies were performed as follows to determine the related substances of diisobutyryl epinephrine due to hydrolysis in Diisobutyryl Epinephrine Sublingual Film (DESF) by HPLC. The four formulations shown in Table 1B were used.

Required Materials and Equipment

1.1 Epinephrine Reference Standard

1.2 Purified Water, or equivalent

1.3 Acetonitrile (ACN), HPLC Grade or equivalent

1.4 Ammonium Formate (Am F), ACS grade or equivalent

1,5 Formic Acid (FA), ACS grade or equivalent

1.6 Phosphoric Acid (H₃PO₄), ACS grade or equivalent

1.7 Analytical Balance, capable of accurately weighing to the nearest 0.01 mg

1.8 HPLC with UV/PDA Detector

1.9 ACE HILIC-N 1.7 μm, 3×100 mm, p/n HILN-17-1003U HPLC column

1.10 Waters Empower Data Acquisition System or equivalent

1.11 Wrist-Action shaker

1.12 Ultrasonic bath

1.13 Glass Pipettes, Class “A”

1.14 Volumetric Flasks, Class “A”

1.15 Plastic Syringe

1.16 0.45 μm Nylon syringe filter

Wash Solutions were Prepared as Follows:

1.17 Mobile Phase A—0.1% Formic Acid in 200 mM Ammonium Formate:
Accurately weigh 12.6 g of Ammonium Formate and dissolve in 1 L of purified water measured with Class “A” glassware. Transfer 1.0 mL of FA into the buffer solution and mix well. Degas before use.

1.1.8 Mobile Phase B—0.1% Formic Acid in Acetonitrile:
Transfer 1.0 mL of FA into 1 L of ACN measured with Class “A” glassware and mix well. Degas before use.

1,19 Diluent A—0.05% H₃PO₄ in 50/50 Water/Acetonitrile:
Combine 1000 mL of purified water with 1000 mL of ACN. Transfer 1.0 mL of H₃PO₄ and mix well. Allow the solution to equilibrate to room temperature before use.

1.20 Diluent B/Column Wash-2-10/90 Water/Acetonitrile:
Combine 100 mL of purified water with 900 mL of ACN. Allow the solution to equilibrate to room temperature before use.

1.21 Needle/Column Wash-1-50/50 Water/ACN:
Combine 500 mL of purified water with 500 mL of ACN and mix well.

1.22 Seal Wash—95/5 Water/ACN:
Combine 950 mL of purified water with 50 mL of ACN and mix well.
   Standard Solutions were Prepared as Follows:

1.23 Epinephrine Stock Standard Solution (120 μg/mL)
Accurately weigh 12 mg (±10%) of the Epinephrine reference standard into a 100.0 mL volumetric flask. Add approximately 50 mL of Diluent A. Sonicate for 5 minutes or until dissolved completely. Dilute to volume with Diluent A and mix well.

1.24 Working Standard Solution 0.5% (1.2 μg/mL Epinephrine)
Pipet 1.0 mL of Epinephrine stock standard solution into a 100 mL volumetric flask and bring to volume with Diluent B and mix well.

1.25 Epinephrine LOQ Solution 0.05% (0.12 μg/mL Epinephrine)
Pipet 2.0 mL of Working Standard Solution to a 20 mL volumetric flask. Dilute to volume with Diluent B and mix well. Prepare fresh on day of analysis.

### Preparation of Samples

1.26 Impurities Stock Sample Solution
Weigh film samples and transfer to volumetric flasks as specified in the table below. Add approximately 50% of the volume of diluent A and mechanically shake to dissolve for 30 minutes. Dilute to volume with diluent A and mix well.

TABLE 5
DESF Stock Sample Solution Preparation Scheme

| | | | | Diisobutyryl |
|---|---|---|---|---|
| DESF Label | Eugenol LC | | Volumetric | Epinephrine |
| Claim (mg/film) | (mg/film) | # of Film | Flask (mL) | (mg/mL) |
| 6 | 8 | 6 | 50 | 0.72 |
| 12 | 16 | 6 | 100 | 0.72 |
| 18 | 12 | 8 | 200 | 0.72 |
| 24 | 16 | 6 | 200 | 0.72 |

1.27 Impurities Working Sample Solution
Pipet 10.0 mL of impurities sample stock solution into a 25 mL volumetric flask and bring to volume with Acetonitrile and mix well. Filter the solution through a 0.45 μm Nylon syringe filter into HPLC vials, discarding at least 1 mL of the filtrate.

1.28 Placebo Stock Solution
Transfer one (1) placebo film strip into a 20 mL volumetric flask. Add Diluent A to about half the volume and mechanically shake to dissolve for 30 minutes. Dilute to volume with Diluent A and mix well.

1.29 Placebo Working Sample Solution
Pipet 5.0 mL of placebo stock solution into a 25 mL volumetric flask and bring to volume with Acetonitrile and mix well. Filter the solution through a 0.45 μm Nylon syringe filter into HPLC vials, discarding at least 1 mL of the filtrate.

1.30 Diluent Blank
Transfer an aliquot of the Diluent B used for sample and working standard preparation into a HPLC vial for analysis.

### 2 Calculations

2.1 Standard Concentration (μg/mL):

$$WSA ( WSB ) Conc . ( μ g / mL ) = \frac{Std Wt ( mg )}{{D}_{S} ( mL )} ⨯ P ⨯ 1000 ( μg / mg )$$

Where:

### P=Reference Standard Purity

Std Wt=Weight of the Epinephrine reference standard
D_{S}=Standard dilution

2.2 Quantification of Related Substances:

$$Hydrolysis Imp ( \% LC ) = \frac{{A}_{U}}{{A}_{S}} ⨯ WSA Conc . ( μ g / mL ) ⨯ \frac{{D}_{U} ( mL )}{N} ⨯ \frac{1}{LC ⨯ RRF} ⨯ \frac{100 \%}{1000 ( μ g / mg )}$$

Where:

A_{U}=Diisobutyryl Epinephrine related Substance peak area in the sample
A_{S}=Average Epinephrine peak area in all WSA injections
D_{U}=Sample dilution
N=Number of strips in the sample preparation
LC=Label Claim of Diisobutyryl Epinephrine Film expressed as mg/strip

### RRF=Relative Response Factor

100%=Percentage conversion

### Reporting

2.3 Report individual Hydrolysis impurity results ≥0.05% as found.
2.3.1 LOD=0.03% (0.07 μg/mL)
2.3.2 LOQ=0.05% (0.12 μg/mL).

2.4 Report individual Hydrolysis impurity results <0.05% as “<0.05%”.

2.5 Exclude individual impurity results <0.05% from total impurity calculations.

2.6 Report individual Hydrolysis impurity results (% LC) to two decimals.

2.7 RRTs and RRFs for known impurities are listed in Table 4.

TABLE 6
RRT's and RRFs for Excipients and Impurity Peaks

| | Retention Time | RRT to | Relative Response |
|---|---|---|---|
| Peak Name | (min) | Epinephrine | Factor (RRF) |
| MIE | 3.0 | 0.45 | 0.5240¹ |
| Epinephrine | 6.6 | 1.0 | 1 |

¹Use 1/RRF in Empower “Relative Response Factor” field; ref: ADR-0121-01

Stability was measured after 6 months. The data trend predicts ˜95% assay remaining at 24 months.

After 6 months, at 25° C. show no significant change (<5%) from T=0. Differentiation was observed at 40° C. Significant (>5%) change observed at 6 months 40° C. in Form 1 and Form 3.

In terms of stability, formula 4 was ranked most stable, with formula 2 ranked second, formula 3 ranked third, and formula 1 ranked fourth.

TABLE 7
Assay Change Rate

| Assay Change | | |
|---|---|---|
| Rate | | |
| % LC/month | | |
| (trendline | | |
| slope) | 25/60 | 40/75 |
| Form 1 | 0.22 | 1.22 |
| Form 2 | 0.24 | 0.67 |
| Form 3 | 0.20 | 0.81 |
| Form 4 | 0.14 | 0.46 |

### Clinical Studies

The Phase 1 randomized, single-ascending dose (SAD) study was performed with Formulations 1, 2, 3 and 4 in order to assess safety, tolerability, PK, and PD profiles. The study was conducted in Canada pursuant to a clinical trial application approved by Health Canada. Subjects participating in the trial received, in ascending fashion, sublingually administered doses of Formulations 1, 2, 3 and 4. The four formulation compositions were varied to assess critical absorption factors including drug loading and the use of inventive excipients designed to influence absorption, stability, and the conversion of a prodrug. A target formulation (“formulation #2”) was designed as the lead candidate for the study.

### Study Highlights

Key clinical measures for comparability to existing autoinjectors (Cₘₐₓ, Tₘₐₓ, and area under the curve, or AUC) were within expected ranges for formulations 1, 2 (target), and 4
The Tₘₐₓ for multiple formulations fell into a narrower range compared to published data for autoinjectors
Observed PD values were comparable to existing autoinjector data
Formulations 1, 2, 3 and 4 was generally well tolerated with no serious adverse events

TABLE 8
Prodrug Profiles

| | Epinephrine prodrug sublingual film)12 mg | | | | | | |
|---|---|---|---|---|---|---|---|
| Study Results | | 2 | | | Comparable Data from Previous & Published Studies | | |
| Film Formulation # | 1 | (Target) | 3 | 4 | EpiPen ®¹ | EpiPen ®² | Auvi-Q ®¹ |
| Dosings (n) | 6 | 8 | 6 | 7 | 135 | 10 | 67 |
| Cₘₐₓ (pg/mL) | 552 | 762 | 164 | 307 | 518 | 341 | 484 |
| AUC 0-t (hr*pg/mL) | 634 | 603 | 329 | 303 | 560 | 328 | 526 |
| Median Tₘₐₓ (minutes) | 15 | 15 | 20 | 10 | 10 | 22 | 20 |
| Tₘₐₓ range (minutes) | 15-25 | 10-35 | 20-50 | 5-50 | 4-60 | 5-90 | 5-60 |

¹Dworaczyk D., Hunt A., Presented at American Academy of Allergy, Asthma, Immunology (AAAAI) National Conference, Mar. 16, 2020. brynpharma.com/media/content/docs/comparative-delivery-poster.pdf;
²Aquestive Therapeutics, Study 160455, on file.

This study indicated that Formulations 1, 2, 3 and 4 were absorbed and rapidly converted to epinephrine with an observed median Tₘₐₓ of 15 minutes and an observed geometric mean Cₘₐₓ of 762 μg/mL for the target formulation. This is comparable to published study results for both EpiPen® and Auvi-Q®. In addition, the target formulation had similar median Tₘₐₓ values at lower dose strengths (15 minutes and 17.5 minutes for the 6 mg and 9 mg doses respectively). From the study results, Aquestive plans on continuing development of the target formulation. See, e.g., Dworaczyk D., Hunt A., Presented at American Academy of Allergy, Asthma, Immunology (AAAAI) National Conference, Mar. 16, 2020. brynpharma.com/media/content/docs/comparative-delivery-poster.pdf; Aquestive Therapeutics, Study 160455, on file; Dworaczyk D., Hunt A., J Allergy Clin Immunol Pract. 2021; 147(2):(2 suppl) AB241 Presented at American Academy of Allergy, Asthma and Immunology (AAAAI) National Conference; Mar. 16, 2020; Accessed Mar. 2, 2021; Worm M et al. Clin Transl Allergy. 2020:10:21; Duvauchelle T et al. J Allergy Clin Immunol Pract. 2018; 6(4):1257-1263;¹ Breuer C et al. Eur J Clin Pharmacol. 2013; 69:1303-1310; Edwards E S et al. Ann Allergy Asthma Immunol. 2013; 111(2):132-137.

Safety data indicated that Formulations 1, 2, 3 and 4 were generally well tolerated with no serious adverse events (SAE's), significant medical events, or treatment-related severe adverse events reported within the trial. All treatment-emergent adverse events (TEALs) deemed at least possibly related were mild to moderate in nature across cohorts.

The PD markers measured changes from baseline in heart rate, systolic blood pressure, and diastolic blood pressure. The values observed suggest a comparable effect from Formulations 1, 2, 3 and 4 and what is expected following autoinjector treatments on these metrics in healthy volunteers.

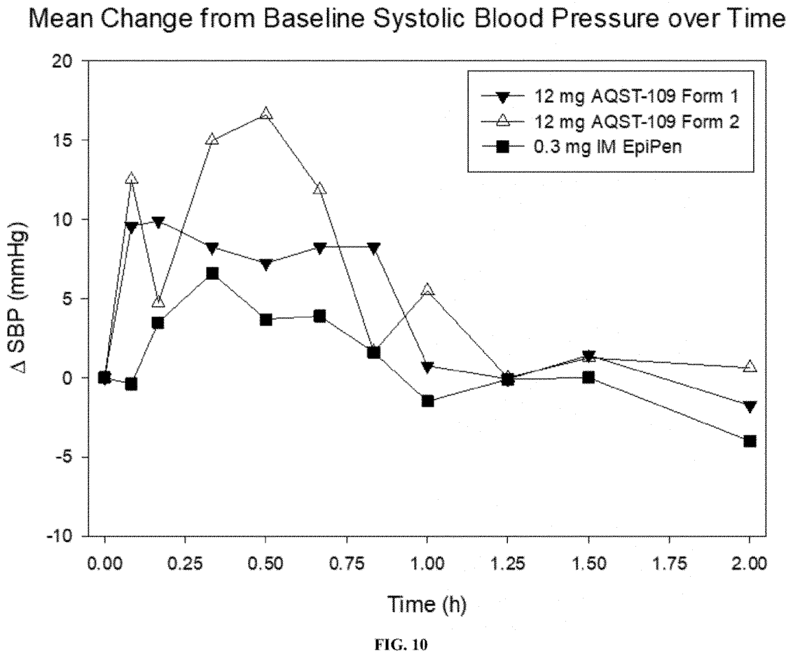

Referring to FIG. 10, mean change in systolic blood pressure is shown for Formulations 1 and 2 vs. Epipen®. It is generally accepted that subjects should see a change in systolic blood pressure over time after the administration of epinephrine. Formulations 1 and 2 shows a similar change from baseline systolic blood pressure when compared to EpiPen® data. This pharmacodynamic ‘marker’ provides a secondary indication that Formulations 1 and 2 are each working as intended after administration.

TABLE 9
Adverse Events Following 12 mg Dose
Adverse events (AE) were measured following 12 mg
dose administration of Formulations 1-4.
Most AE were of mild severity and there have been no serious adverse events.

| | Formulation 1 | | Formulation 2 | | Formulation 3 | | Formulation 4 | |
|---|---|---|---|---|---|---|---|---|
| | n = 6 | | n = 8 | | n = 6 | | n = 7 | |
| | Mild | Moderate | Mild | Moderate | Mild | Moderate | Mild | Moderate |
| Gen. | 13 | 0 | 31 | 0 | 13 | 0 | 14 | 0 |
| Administration | | | | | | | | |
| and Site | | | | | | | | |
| Conditions | | | | | | | | |
| GI | 2 | 0 | 2 | 1 | 0 | 0 | 1 | 0 |
| CV | 1 | 0 | 2 | 0 | 0 | 0 | 0 | 0 |
| Other | 1 | 0 | 3 | 0 | 1 | 0 | 0 | 0 |

### Example 9: Impact of Drug Load in Hydrolysis Studies

Referring to the formulations in Table 1B, the following studies on drug load and sodium fluoride appear to have strongest impact on hydrolysis. The rate of growth of the hydrolysis product is largest in formulations 3 and 1; where formulation 3 omits sodium fluoride, and formulation 1 contains sodium fluoride but has a lower drug load. These aspects demonstrate that the absence of sodium fluoride and lower drug load both contribute to an increase in the hydrolysis product.

### Hydrolysis Product

According to the data, at 25° C. trend predicts ˜4% at 24 months in Formula 1 & 2-3% in Formula 2/Form 4.

TABLE 10
% LC/month

| MIE Growth | | |
|---|---|---|
| (% LC/month) | 25/60 | 40/75 |
| Form 1 | 0.16 | 0.60 |
| Form 2 | 0.09 | 0.38 |
| Form 3 | 0.22 | 0.64 |
| Form 4 | 0.08 | 0.29 |

Similar results observed through three months in clinical films

### Epinephrine

25° C. through 6 months is below quantitation limit (0.05%).

40° C. through 6 months is at 0.5% in Form 1

### Example 10

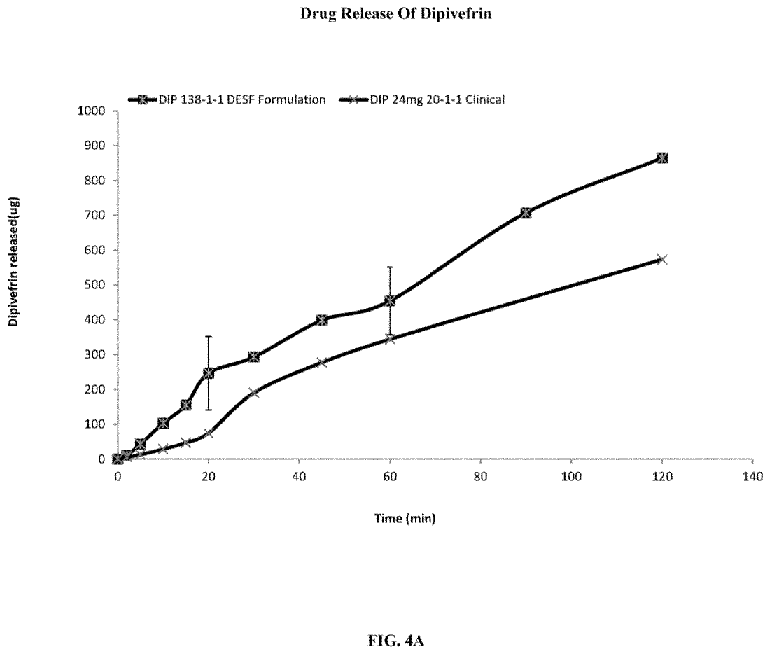

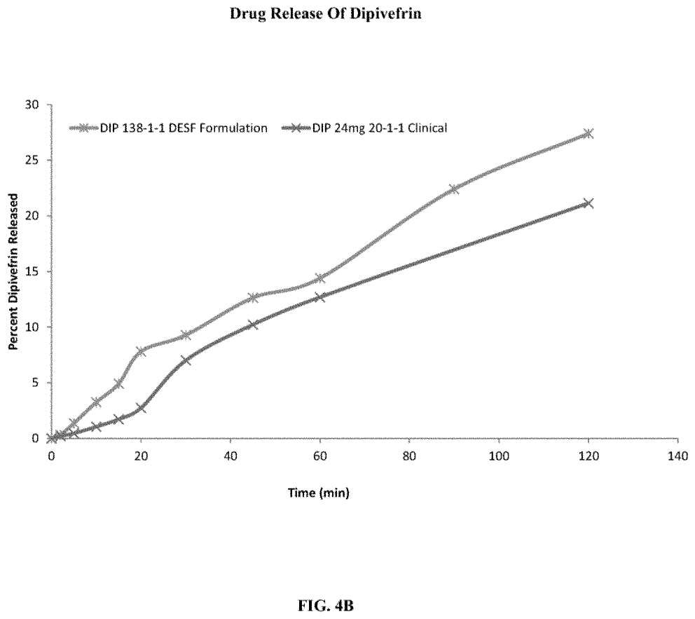

Referring to FIGS. 4A and 4B, the data shows improved drug release of dipivefrin from both platform films.

### Example 11

A dipivefrin film formulation was manufactured using the excipients and process used for the manufacture of DESF. The stability achieved with the DESF platform is shown in FIG. 8. The shows that excellent stability is achieved even at accelerated temperatures. The data is summarized in the table below. One of the hydrolysis product is monopivaloyl epinephrine (MPE).

TABLE 11
Stability of dipivefrin in DESF platform formulation (138-1-1)

| | | | | Unknown Degradants (% LC) | | | | | | | |
|---|---|---|---|---|---|---|---|---|---|---|---|
| | Stability | Epi | MPE | RRT | RRT | RRT | RRT | RRT | RRT | RRT | RRT |
| Lot | condition | % | (%) | 0.94 | 1.16 | 1.34 | 1.40 | 1.66 | 1.79 | 1.82 | 1.90 |
| 138-1-1 | Initial | <0.05 | 0.34 | ND | 0.14 | <0.05 | ND | ND | 0.06 | ND | ND |
| | 3M, 25° C. | ND | 0.4 | ND | 0.14 | 0.12 | ND | ND | ND | ND | ND |
| | 3M 40° C. | ND | 0.8 | ND | 0.08 | 0.09 | ND | ND | ND | 0.12 | ND |

### Example 12

Degradation can occur via several pathways, including primarily trans-esterification and hydrolysis for example. A stabilizer can protect a composition against a degradation pathway or a combination of these pathways as shown. An Arrhenius-based model was developed to projected degradation rates for DESF impurities. The Arrhenius equation was used to study the relationship between reaction rate and temperature. The model accurately predicted the monoisobutryryl epinephrine (PD-15) degradant levels and correlated well with real time data generated under ICH stability conditions. The projected degradant levels were measured as % LC at the end of 6 month at 40° C., 12-month at 30° C., and 25 month at 25° C. storage. The projected shelf life was analyzed for 3-5 years storage at 25° C. All degradants remained within specification limits at the end of each storage period. The first degradant PD-15 is expected to fail at 25° C. storage at 4 years (range of 3.1-5.2 years). The degradation rate at 25° C. presented as % LC/day.

TABLE 12
Impurity Degradation Rates

| | | | % LC | | | | | | |
|---|---|---|---|---|---|---|---|---|---|
| | | | 24M | 12M | 6M | Deg Rate | Shelf-Life Projection (years) | | |
| Degradant | Initial | Limit | 25 C. | 30 C. | 40 C. | (% LC/day) | Target | LCL | UCL |
| Epi [symbol] | 0 | 0.5 | 0.20 | 0.20 | 0.40 | 2.75 × 10⁻⁴ | 4.99 | 3.98 | 6.26 |
| •Monoisobutryryl [symbol] | 0.77 | 4 | 2.37 | 2.34 | 3.55 | 2.19 × 10⁻³ | 4.04 | 3.13 | 5.19 |
| Epinephrine [symbol] | | | | | | | | | |
| RRT 1.79 | 0 | 0.5 | 0.20 | 0.22 | 0.49 | 2.73 × 10⁻⁴ | 5.01 | 4.24 | 5.92 |
| RRT 2.78 | 0 | 0.5 | 0.15 | 0.16 | 0.34 | 2.01 × 10⁻⁴ | 6.80 | 5.39 | 8.59 |
| RRT 3.10 | 0 | 0.5 | 0.12 | 0.11 | 0.19 | 1.60 × 10⁻⁵ | 8.56 | 7.08 | 10.35 |

[symbol]

indicates data missing or illegible when filed

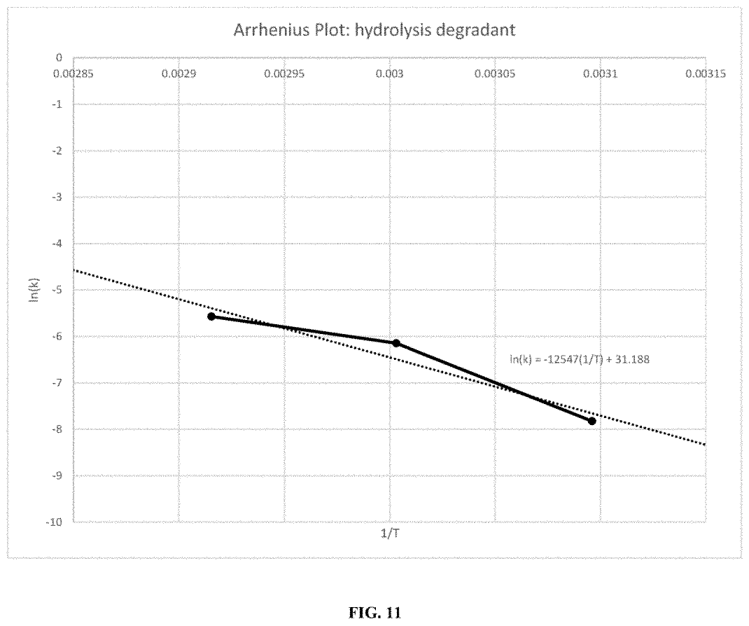

Referring to FIG. 11, and as reflected in Table 13 below, the Arrhenius Equation predicted the reaction rate as a function of temperature of the monoisobutryryl epinephrine hydrolysis product. Plotting the natural log of the calculated reaction rates against the inverse of the temperature yields a straight line with y-intercept ln(A) and slope −Eₐ/R. In this equation, A and R are constants, and Eₐ is the activation energy for the reaction. Inputting a temperature into the equation allows the calculation of the reaction rate at that temperature.

$$ln ( k ) = ln ( A ) - \frac{{E}_{a}}{R} ( \frac{1}{T} )$$

TABLE 13
Monoisobutryryl Epinephrine Hydrolysis Reaction
Rate as a Function of Temperature

| | Reaction Rate | | |
|---|---|---|---|
| Temp | (k) | 1/T | ln(k) |
| 50 | 0.0003993 | 0.003096 | −7.82569 |
| 60 | 0.0021464 | 0.003003 | −6.14396 |
| 70 | 0.0038065 | 0.002915 | −5.57104 |

Other aspects, embodiments, and features will be apparent from the following description, the drawings, and the claims.

What is claimed is:

1. A pharmaceutical composition with enhanced dissolution comprising
an active ingredient,
a film forming polymer including a starch ether, and
a desiccant.

2. The composition of claim 1, wherein the starch ether is a hydroxyalkyl ether of a starch.

3. The composition of claim 2, wherein the hydroxyalkyl ether of a starch is a hydroxypropyl ether of a starch.

4. The composition of claim 1, wherein the film forming polymer is a pea starch.

5. The composition of claim 1, wherein the desiccant includes a silica.

6. The composition of claim 1, wherein the desiccant includes a fumed silica or a mesoporous silica.

7. The composition of claim 1, wherein the film forming polymer and the desiccant have a ratio of 10:1 to 2:1 by weight.

8. The composition of claim 1, wherein the active ingredient comprises 0.1% to 80% of the composition by weight.

9. The composition of claim 1, further comprising a stabilizer.

10. The composition of claim 9, wherein the stabilizer includes a chelating agent.

11. The composition of claim 1, further comprising an antioxidant.

12. The composition of claim 9, wherein the stabilizer includes an ion exchange resin.

13. The composition of claim 12, wherein the ion exchange resin is a cation exchange resin.

14. The composition of claim 1, further comprising a permeation enhancer.

15. The composition of claim 1, further comprising a permeation enhancer includes an adrenergic receptor interacter.

16. The composition of claim 13, wherein the permeation enhancer includes eugenol.

17. The composition of claim 1, further comprising a processing solvent.

18. The composition of claim 16, wherein the processing solvent is an organic processing solvent.

19. The composition of claim 16, wherein the processing solvent includes one or more of ethanol, acetone, acetonitrile, t-butanol, methanol, 1-propanol, isopropanol, tetrahydrofuran, acetaldehyde, dioxane, or methylisocyanide.

20. The composition of claim 16, wherein the processing solvent includes at least 20% ethanol.

21. The composition of claim 16, wherein the processing solvent includes at least 30% ethanol.

22. The composition of claim 16, wherein the processing solvent includes at least 40% ethanol.

23. The composition of claim 16, wherein the processing solvent includes at least 50% ethanol.

24. The composition of claim 1, further comprising a plasticizer.

25. The composition of claim 23, wherein the plasticizer includes a polyol.

26. The composition of claim 23, wherein the plasticizer includes a pentatol.

27. The composition of claim 23, wherein the plasticizer includes a sucralose; sugar alcohols such as sorbitol, mannitol, xylitol.

28. The composition of claim 1, further comprising a viscosity builder.

29. The composition of claim 27, wherein the viscosity builder includes gelatin, xantham gum, ethyl cellulose, hydroxy propyl cellulose, methyl cellulose, microcrystalline cellulose, chitosan, natural gums, polyvinyls, crosslinked polymers, or other synthetic polymers.

30. The composition of claim 1, further comprising a surfactant.

31. The composition of claim 29, wherein the surfactant includes Labrasol®.

32. The composition of claim 29 wherein the surfactant includes GMO.

33. The composition of claim 1, further comprising an esterase inhibitor.

34. The composition of claim 32, wherein the esterase inhibitor includes NaF.

35. The composition of claim 1, further comprising a sweetener.

36. The composition of claim 34, wherein the sweetener includes sucralose.

37. The composition of claim 32, wherein the sweetener includes Magnasweet™.

38. The composition of claim 1, further comprising a flavoring agent.

39. The composition of claim 1, further comprising a coloring agent.

40. A pharmaceutical composition for delivering a pharmaceutical composition with enhanced stability comprising an active ingredient,
a pH modifier including HCl, and
a plasticizer including a non-reducing sugar.

41. The composition of claim 37, wherein the non-reducing sugar is a polyol.

42. The composition of claim 37, wherein the non-reducing sugar is a pentatol.

43. The composition of claim 37, wherein the non-reducing sugar is a xylitol.

44. The composition of claim 1, wherein the pH modifier results in a formulation pH of 2.5 to 3.5 and plasticizer has a ratio of 1:20 to 1:8 by weight.

45. A pharmaceutical film product comprising
an active ingredient,
a stabilizer,
a plasticizer, and
the film product has a small volume disintegration value in the range of about 1 to about 240 seconds as measured according to a small volume disintegration assay.

46. The pharmaceutical film product of claim 42, wherein the film product has a small volume disintegration time in the range of about 2 to about 30 seconds.

47. The pharmaceutical film product of claim 42, wherein the film product has a small volume disintegration time in the range of about 2 to about 10 seconds.

48. A method of making a pharmaceutical composition with enhanced stability comprising forming a composition having a dissolution profile in the range of about 1 to about 60 seconds as measured according to a small volume disintegration assay.

49. The method of claim 45, wherein the film product has a partial immersion dissolution value in the range of about 2 to about 30 seconds as measured according to a small volume disintegration assay.

50. The pharmaceutical film product of claim 42, wherein the film product has a partial immersion dissolution value in the range of about 2 to about 10 seconds as measured according to a small volume disintegration assay.

51. A method of making a pharmaceutical formulation with an enhanced dissolution rate comprising
providing an active ingredient
incorporating a desiccant including mesoporous silica and
applying a film forming polymer including a pea starch.

52. A method of making a pharmaceutical formulation with enhanced stability comprising
providing an active ingredient,
incorporating a pH modifier that results in formulation pH of 2.5 to 3.5, and
incorporating a plasticizer including xylitol.

53. A method of stabilizing transmucosally delivered epinephrine comprising:
administering a pharmaceutical composition including
an active ingredient,
a pH modifier results in a formulation pH of 2.5 to 3.5,
a desiccant including mesoporous silica, and
a plasticizer including xylitol; and
achieving an effective plasma concentration of a pharmaceutically active form of epinephrine in less than 1 hour.

54. The method of claim 49, wherein the pH modifier is HCl.

55. The method of 50, wherein the pH modifier is HCl.

56. The composition of claim 1, wherein the active ingredient includes a prodrug of epinephrine.

57. The composition of claim 56, further comprising a degradant.

58. The composition of claim 57, wherein the degradant is a hydrolysis product of the prodrug of epinephrine.

59. The composition of claim 57, wherein the degradant is a portion of the delivered active ingredient delivered to a subject.

60. The composition of claim 57, wherein the degradant level is present at about 3.5% or more at the end of 6 months.

61. The composition of claim 58, wherein the degradant level is present at about 2.3% or more at the end of 12 months.

62. The composition of claim 58, wherein the degradant level is present at about 2.4% or more at the end of 24 months.

63. The composition of claim 58, wherein the degradant has a degradation rate of about 2.2×10⁻³%.

64. The composition of claim 58, wherein the degradant maintains a shelf life for storage at 25° C. for at least 3 years.

65. The composition of claim 58, wherein the degradant maintains a shelf life for storage at 25° C. for at least 4 years.

66. The composition of claim 58, wherein the degradant maintains a shelf life for storage at 25° C. for at least 5 years.

67. The composition of claim 58, wherein the rate of degradant growth is substantially unchanged for at least 3 months.

68. The composition of claim 58, wherein the rate of degradant growth is substantially unchanged for at least 5 months.
